(19) United States
(12) Patent Application Publication
(10) Pub. No.: US 2023/0242720 A1
(43) Pub. Date: Aug. 3, 2023

(54) MODULUS MODIFIERS AND FILMS THEREOF

(71) Applicants: ZYMERGEN INC., Emeryville, CA (US); SUMITOMO CHEMICAL COMPANY, LIMITED, Tokyo (JP)

(72) Inventors: Arjan ZOOMBELT, San Mateo, CA (US); John McNAMARA, EI Sobrante, CA (US); Joachim RITTER, San Rafael, CA (US); Michael William Angus MACLEAN, Oakland, CA (US); Kelby HULL, Emeryville, CA (US); Patrick HOMYAK, Oakland, CA (US); Peter Lucas POLIAK, Emeryville, CA (US); Kazuya MATSUI, Tokyo (JP); Satoshi OKAMOTO, Tokyo (JP)

(21) Appl. No.: 18/003,611

(22) PCT Filed: Jun. 29, 2021
(86) PCT No.: PCT/US2021/039670

§ 371 Date: Dec. 28, 2022

Related U.S. Application Data
(60) Provisional application No. 62/705,479, filed on Jun. 29, 2020.

(51) Int. Cl. C08J 5/18; C07D 241/08; C07D 487/14; C07J 41/00

(52) U.S. Cl. CPC C08J 5/18; C07D 241/08; C07D 487/14; C07J 41/0011; C08J 2379/08; C08J 2377/06

(57) ABSTRACT

The present disclosure relates to compositions derived from bioreachable molecules, such as amino acids and/or steroids. In particular, the composition can be a monomer, a polymer, or a copolymer derived from an amino acid dimer. Such compositions can optionally include a functionalized steroid.

### TECHNICAL FIELD

The present disclosure relates to compositions derived from bioreachable molecules, such as amino acids and/or steroids. In particular embodiments, the composition can be a monomer, a polymer, or a copolymer derived from an amino acid dimer. Such compositions can optionally include a functionalized steroid.

### BACKGROUND

Polymeric resins are generally high production chemicals with varying degrees of toxicity and environmental effects.

### SUMMARY

The present disclosure relates to compositions derived from bioreachable molecules having origin from biological resources. Illustrative bioreachable molecules include amino acids and steroids, which can be produced by microbes through fermentation and/or biotransformation. Such bioreachable molecules can be structurally modified to provide, e.g., cyclic dimers, which in turn can be further chemically functionalized with other moieties to provide cyclic derivatives. In addition, steroids can be structurally modified to provide functionalized steroids. The resulting cyclic derivatives and/or functionalized steroids can be employed as monomers to provide polymers or copolymers.

Accordingly, in a first aspect, the present disclosure encompasses a composition including a structure having formula (I):

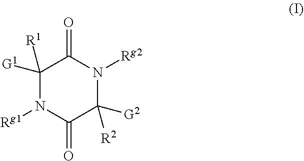

or a salt thereof, wherein each of G¹ and G² includes a reactive moiety (e.g., any described herein). In some embodiments, each of G¹ and G² includes, independently, hydroxyl, carboxyl, amino, amido, optionally substituted cyclic anhydride, or optionally substituted cyclic imide. In other embodiments, each of R¹ and R² is, independently, H or optionally substituted alkyl. In yet other embodiments, each of R^{g1} and R^{g2} is, independently, optionally substituted alkyl, optionally substituted aryl, optionally substituted alkaryl, or optionally substituted aralkyl; in which R^{g1} and G¹, taken together with the nitrogen to which R¹ is bound, can optionally form an optionally substituted heterocyclyl; and in which R^{g2} and G² taken together with the nitrogen to which R^{g2} is bound, can optionally form an optionally substituted heterocyclyl.

In some embodiments, the composition includes a structure having formula (Ia), (Ib), or (Ic):

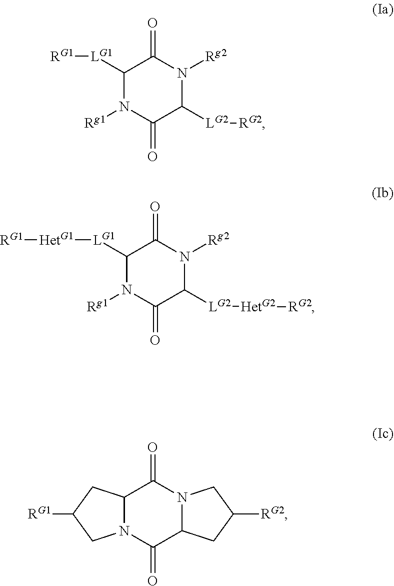

or a salt thereof. In particular embodiments, each of L^{G1} and L^{G2} is, independently, a covalent bond, an amide bond, optionally substituted alkylene, optionally substituted alkenylene, optionally substituted heteroalkylene, optionally substituted heterocyclyldiyl, or optionally substituted (heterocyclyl)(alkyl)ene. In other embodiments, each of R^{G1} and R^{G2} is, independently, hydroxyl, optionally substituted hydroxyalkyl, optionally substituted hydroxyaryl, carboxyl, amino, optionally substituted aminoalkyl, optionally substituted aminoaryl, amido, optionally substituted cyclic anhydride, or optionally substituted cyclic imide. In yet other embodiments, each of Het^{G1} and Het^{G2} is, independently, optionally substituted heterocyclyldiyl or optionally substituted (heterocyclyl)(alkyl)ene. In other embodiments, the composition includes a structure having formula (Id), (Ie), (If), (Ig), (Ih), (Ii), (Ij), (Ik), or a salt thereof (e.g., as described herein).

In a second aspect, the present disclosure encompasses a composition including a structure having formula (II):

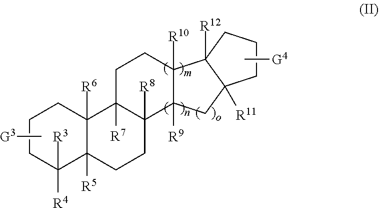

or a salt thereof. In some embodiments, each of G³ and G⁴ includes a reactive moiety (e.g., any described herein). In other embodiments, each of R³, R⁴, R⁵, R⁶, R⁷, R⁸, R⁹, R¹⁰, R¹¹, and R¹² is, independently, H or optionally substituted alkyl; each of m and n is, independently, an integer of from 0 to 1; and o is an integer of from 0 to 2. In yet other embodiments, the composition includes a structure having formula (IIa), (IIb), (IIc), (IId), or a salt thereof (e.g., as described herein).

In particular embodiments (e.g., of formula (II)), each of G³ and G⁴ includes, independently, hydroxyl, carboxyl, amino, amido, optionally substituted cyclic anhydride, or optionally substituted cyclic imide. In other embodiments, G³ is -L^{G1}-R^{G1}, and G⁴ is -L^{G2}-R^{G2} (e.g., as described herein).

In a third aspect, the present disclosure encompasses a method of making a composition (e.g., any described herein, including a composition having a structure of formula (I), (Ia), (Ib), (Ic), (Id), (Ie), (If), (Ig), (Ih), (Ii), (Ij), or (Ik)). In some embodiments, the method includes providing a first amino acid and a second amino acid; and forming a dimer between the first and second amino acids.

In particular embodiments, the first and second amino acids are selected from any herein. In other embodiments, the first and second amino acids are selected from glycine, vinylglycine, 2-allylglycine, alanine, valine, leucine, isoleucine, methionine, phenylalanine, 4-aminophenylalanine, tyrosine, tryptophan, serine, threonine, asparagine, glutamine, cystine, homocystine, cysteine, homocysteine, selenocysteine, proline, hydroxyproline, arginine, histidine, lysine, aspartic acid, glutaminic acid, 4-(2-amino-2-carboxyethyl)-1,2-benzenedicarboxylic acid, 1-aminopropane-1,2,3-tricarboxylic acid, and 4-amino-1,2,4-butanetricarboxylic acid. In yet other embodiments, the first and second amino acids are selected from the group tyrosine, serine, threonine, and hydroxyproline.

In a fourth aspect, the present disclosure encompasses another method of making a composition (e.g., any described herein, including a composition having a structure of formula (I), (Ia), (Ib), (Ic), (Id), (Ie), (If), (Ig), (Ih), (Ii), (Ij), or (Ik)). In some embodiments, the method includes providing a first amino acid and a second amino acid; forming a dimer between the first and second amino acids; and hydroaminating an optionally substituted alkenyl group (e.g., a vinyl group) present on the first and second amino acids. In particular embodiments, the first and second amino acids include one or more optionally substituted alkenyl groups. In other embodiments, the first and second amino acids are selected from vinylglycine, 2-allylglycine, alkenylglycine, O-allyltyrosine, O-alkenyltryrosine, allyltryptophan, alkenyltryptophan, allylphenylalanine, and alkenylphenylalanine. In yet other embodiments, hydroaminating can include use of a nitrogen-containing reactant in the presence of the optionally substituted alkenyl group. Exemplary, non-limiting nitrogen-containing reactants include an amine, such as NR^{N1}R^{N2}R^{N3}, in which each of R^{N1}, R^{N2}, and R^{N3} is, independently, H, optionally substituted alkyl, optionally substituted alkenyl, optionally substituted alkynyl, optionally substituted aryl, or a leaving group, such as an ester group.

In a fifth aspect, the present disclosure encompasses a composition including a structure having formula (III):

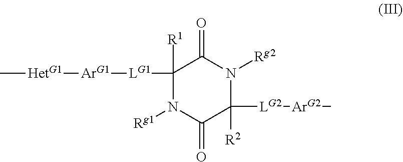

or a salt thereof. In some embodiments, at least one of Ar^{G1}, Ar^{G2}, or Het^{G1} includes a reactive moiety or a reaction product of a reactive moiety (e.g., any reactive moiety herein). In other embodiments, at least one of Ar^{G1}, Ar^{G2}, or Het^{G1} includes, independently, hydroxyl, carboxyl, amino, amido, an amide bond, an ester bond, oxy, carbonyl, optionally substituted cyclic anhydride, or optionally substituted cyclic imide.

In other embodiments, each of R¹ and R² is, independently, H or optionally substituted alkyl; and each of R^{g1} and R^{g2} is, independently, optionally substituted alkyl, optionally substituted aryl, optionally substituted alkaryl, or optionally substituted aralkyl. In yet other embodiments, each of L^{G1} and L^{G2} is a linker (e.g., any herein, such as a covalent bond, an amide bond, optionally substituted alkylene, optionally substituted alkenylene, optionally substituted heterocyclyldiyl, or optionally substituted (heterocyclyl)(alkyl)ene). In some embodiments, each of Ar^{G1} and Ar^{G2} is, independently, optionally substituted arylene or optionally substituted (aryl)(alky)lene; and Het^{G1} is optionally substituted heterocyclyldiyl or optionally substituted (heterocyclyl)(alkyl)ene. In yet other embodiments, the composition includes a structure having formula (IIIa) or a salt thereof (e.g., as described herein).

In a sixth aspect, the present disclosure encompasses a composition including a structure having formula (IV):

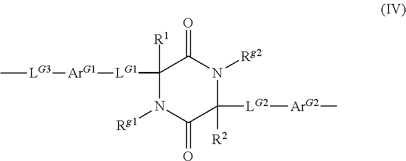

or a salt thereof. In some embodiments, at least one of Ar^{G1}, Ar^{G2}, or L^{G3} includes a reactive moiety or a reaction product of a reactive moiety (e.g., any reactive moiety herein). In other embodiments, at least one of L^{G1}, L^{G2}, L^{G3}, Ar^{G1} or Ar^{G2} includes, independently, hydroxyl, carboxyl, amino, amido, an amide bond, an ester bond, oxy, carbonyl, optionally substituted cyclic anhydride, or optionally substituted cyclic imide.

In some embodiments, each of R¹ and R² is, independently, H or optionally substituted alkyl; and each of R^{g1} and R^{g2} is, independently, optionally substituted alkyl, optionally substituted aryl, optionally substituted alkaryl, or optionally substituted aralkyl. In other embodiments, each of L^{G1}, L^{G2}, and L^{G3} is, independently, a covalent bond, an amide bond, optionally substituted alkylene, optionally substituted alkenylene, optionally substituted heteroalkylene, optionally substituted heterocyclyldiyl, or (heterocyclyl)(alkyl)ene; and each of Ar^{G1} and Ar^{G2} is, independently, optionally substituted arylene or optionally substituted (aryl)(alkyl)ene. In yet other embodiments, the composition includes a structure having formula (IVa), (IVb), (IVc), or a salt thereof (e.g., as described herein).

In a seventh aspect, the present disclosure encompasses a composition including a structure having formula (V), (VI), or (VII):

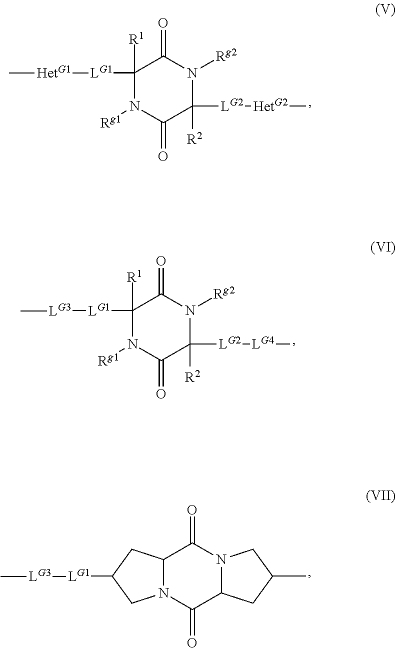

or a salt thereof. In some embodiments, at least one of Het^{G1}, Het^{G2}, L^{G3}, or L^{G4} includes a reactive moiety or a reaction product of a reactive moiety (e.g., any reactive moiety herein). Each of R¹, R², R^{g1}, and R^{g2} can be any described herein. In other embodiments, at least one of Het^{G1}, Het^{G2}, L^{G1}, L^{G2}, L^{G3}, or L^{G4} includes, independently, hydroxyl, carboxyl, amino, amido, an amide bond, an ester bond, oxy, carbonyl, optionally substituted cyclic anhydride, or optionally substituted cyclic imide.

In some embodiments, each of L^{G1}, L^{G2}, L^{G3}, and L^{G4} is, independently, a covalent bond, an amide bond, optionally substituted alkylene, optionally substituted alkenylene, optionally substituted heteroalkylene, optionally substituted heterocyclyldiyl, or (heterocyclyl)(alkyl)ene; and each of Het^{G1} and Het^{G2} is, independently, optionally substituted heterocyclyldiyl or optionally substituted (heterocyclyl)(alkyl)ene. In yet other embodiments, the composition includes a structure having formula (Va), (VIa), (VIb), (VIc), (VIIa), or a salt thereof (e.g., as described herein).

In an eighth aspect, the present disclosure encompasses a composition including a structure having formula (VIII):

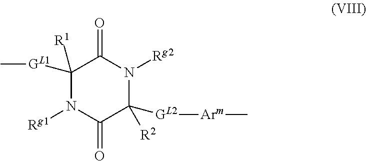

or a salt thereof. In some embodiments, at least one of G^{L1} or G^{L2} includes a reactive moiety or a reaction product of a reactive moiety (e.g., any reactive moiety herein). In some embodiments, each of G^{L1} and G^{L2} includes, independently, hydroxyl, carboxyl, amino, amido, an amide bond, an ester bond, oxy, carbonyl, optionally substituted cyclic anhydride, or optionally substituted cyclic imide. Each of R¹, R², R^{g1}, and R^{g2} can be any described herein, and Ar^{m} is a polymer segment (e.g., any described herein).

In some embodiments, Ar^{m} includes an imide subunit, an amic acid subunit, an amide subunit, an arylene subunit, an arylene ether subunit, an arylene ketone subunit, a urethane subunit, a phthalic anhydride subunit, an aliphatic subunit, a cycloalkyl subunit, an ether subunit, a thioether subunit, a perfluoroalkyl subunit, or a perfluoroalkoxy subunit.

In a ninth aspect, the present disclosure encompasses a genetically modified organism (e.g., any described herein) configured to produce any composition described herein (e.g., a composition including a structure of formula (I), (Ia), (Ib), (Ic), (Id), (Ie), (If), (Ig), (Ih), (Ii), (Ij), (Ik), (II), (IIa), (IIb), (IIc), (IId), (III), (IIIa), (IV), (IVa), (IVb), (IVc), (V), (Va), (VI), (VIa), (VIb), (VIc), (VII), (VIIa), or (VIII), or a salt thereof).

In a tenth aspect, the present disclosure encompasses a film (e.g., any described herein) including any composition described herein (e.g., a composition including a structure of formula (I), (Ia), (Ib), (Ic), (Id), (Ie), (If), (Ig), (Ih), (Ii), (Ij), (Ik), (II), (IIa), (IIb), (IIc), (IId), (III), (IIIa), (IV), (IVa), (IVb), (IVc), (V), (Va), (VI), (VIa), (VIb), (VIc), (VII), (VIIa), or (VIII), or a salt thereof). In some embodiments, the film is an adhesive or a coating.

In an eleventh aspect, the present disclosure encompasses a composite or bulk structure (e.g., any described herein) including any composition described herein (e.g., a composition including a structure of formula (I), (Ia), (Ib), (Ic), (Id), (Ie), (If), (Ig), (Ih), (Ii), (Ij), (Ik), (II), (IIa), (IIb), (IIc), (IId), (III), (IIIa), (IV), (IVa), (IVb), (IVc), (V), (Va), (VI), (VIa), (VIb), (VIc), (VII), (VIIa), or (VIII), or a salt thereof).

In an twelfth aspect, the present disclosure encompasses a fiber or a particle (e.g., any described herein) including any composition described herein (e.g., a composition including a structure of formula (I), (Ia), (Ib), (Ic), (Id), (Ie), (If), (Ig), (Ih), (Ii), (Ij), (Ik), (II), (IIa), (IIb), (IIc), (IId), (III), (IIIa), (IV), (IVa), (IVb), (IVc), (V), (Va), (VI), (VIa), (VIb), (VIc), (VII), (VIIa), or (VIII), or a salt thereof).

In any embodiment herein, G¹ is -L^{G1}-R^{G1}, G² is -L^{G2}-R^{G2}, G³ is -L^{G1}-R^{G1}, and G⁴ is -L^{G2}-R^{G2}, in which each of L^{G1} and L^{G2} is a linker (e.g., any herein) and each of R^{G1} and R^{G2} is a reactive moiety (e.g., any herein). In some embodiments, each of L^{G1} and L^{G2} is, independently, a covalent bond, an amide bond, —NR^{N1}— (in which R^{N1} is H or optionally substituted alkyl), a carbamate bond (e.g., a —O—C(O)—NR^{N1}— bond, in which R^{N1} is H or optionally substituted alkyl), an ester bond, oxy, carbonyl, optionally substituted alkylene, optionally substituted alkenylene, optionally substituted heteroalkylene, optionally substituted heterocyclyldiyl, or optionally substituted (heterocyclyl)(alkyl)ene. In other embodiments, each of L^{G1} and L^{G2} is, independently, optionally substituted arylene or optionally substituted (aryl)(alkyl)ene. In yet other embodiments, each of R^{G1} and R^{G2} is, independently, hydroxyl, optionally substituted hydroxyalkyl, optionally substituted hydroxyaryl, carboxyl, optionally substituted carboxyalkyl, optionally substituted carboxyaryl, amino, optionally substituted aminoalkyl, optionally substituted aminoaryl, amido, optionally substituted amidoalkyl, optionally substituted cyclic anhydride, optionally substituted cyclic imide, optionally substituted alkenyl, optionally substituted alkynyl, optionally substituted epoxy, or optionally substituted (hetero)cycloalkyl.

In any embodiment herein, any linker herein (e.g., each of L^{G1}, L^{G2}, L^{G3}, L^{G4}, G^{L1}, and G^{L2}) is, independently, a covalent bond, an amide bond, —NR^{N1}— (in which R^{N1} is H or optionally substituted alkyl), an ester bond, oxy, carbonyl, optionally substituted alkylene, optionally substituted alkenylene, optionally substituted heteroalkylene, optionally substituted arylene, optionally substituted (aryl)(alkyl)ene, optionally substituted heterocyclyldiyl, or optionally substituted (heterocyclyl)(alkyl)ene.

In any embodiment herein, any reactive moiety (e.g., each of R^{G}, R^{G1}, and R^{G2}) is, independently, hydroxyl, optionally substituted hydroxyalkyl, optionally substituted hydroxyaryl, carboxyl, optionally substituted carboxyalkyl, optionally substituted carboxyaryl, amino, optionally substituted aminoalkyl, optionally substituted aminoaryl, amido, optionally substituted amidoalkyl, optionally substituted cyclic anhydride, optionally substituted cyclic imide, optionally substituted alkenyl, optionally substituted alkynyl, optionally substituted epoxy, or optionally substituted (hetero)cycloalkyl.

In any embodiment herein, the optionally substituted cyclic anhydride has a structure of:

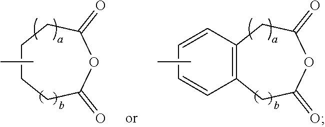

wherein each of a and b, independently, is an integer of from 0 to 3.

In any embodiment herein, the optionally substituted cyclic imide has a structure of:

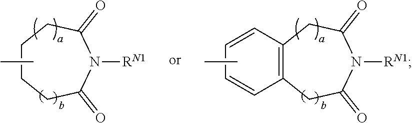

wherein each of a and b, independently, is an integer of from 0 to 3; and wherein R^{N1} is H, optionally substituted alkyl, optionally substituted aryl, or optionally substituted aralkyl.

In any embodiment herein, the composition includes a UV cutoff less than about 450 nm, an optical transmission of at least about 95% at 450 nm, a yellowness index less than about 2, a modulus less than about 12 GPa, and/or an elongation less than about 350%.

In any embodiment herein, the composition is soluble in an organic solvent (e.g., any described herein).

In any embodiment herein, the composition is configured to be biodegradable by one or more microbes or by one or more enzymes (e.g. proteases, hydrolases, cyclic dipeptidases, etc.).

In any embodiment herein, the composition includes a structure of any formula herein (e.g., formula (I), (Ia), (Ib), (Ic), (Id), (Ie), (If), (Ig), (Ih), (Ii), (Ij), (Ik), (II), (IIa), (IIb), (IIc), (IId), (III), (IIIa), (IV), (IVa), (IVb), (IVc), (V), (Va), (VI), (VIa), (VIb), (VIc), (VII), (VIIa), and (VIII)) to modify a modulus (e.g., a Young's modulus) within the composition. In some embodiments, the composition has a modulus less than about 20 GPa, 18 GPa, 15 GPa, 12 GPa, 10 GPa, 8 GPa, 5 GPa, or less. Additional details follow.

### Definitions

By “alkaryl” or “alkylaryl” is meant -Ar-Ak, in which Ar is an optionally substituted arylene, as defined herein, and Ak is an optionally substituted alkyl, as defined herein. The alkaryl group can be substituted or unsubstituted. For example, the alkaryl group can be substituted with one or more substitution groups, as described herein for alkyl and/or aryl. Exemplary unsubstituted alkaryl groups are of from 7 to 16 carbons (C₇₋₁₆ alkaryl), as well as those having an alkyl group with 1 to 6 carbons and an arylene group with 4 to 18 carbons (i.e., (C₁₋₆ alkyl)C₄₋₁₈ aryl).

By “alkenyl” is meant an optionally substituted C₂₋₂₄ alkyl group having one or more double bonds. The alkenyl group can be cyclic (e.g., C₃₋₂₄ cycloalkenyl) or acyclic. The alkenyl group can also be substituted or unsubstituted. For example, the alkenyl group can be substituted with one or more substitution groups, as described herein for alkyl. Exemplary alkenyl groups include, e.g., vinyl (—CH═CH₂), vinylidene (e.g., ═C═CH₂), ethylidene (e.g., ═CH—CH₃), allyl (—CH₂—CH═CH₂), 1-propenyl (—CH═CH—CH₃), methylallyl (—CH₂—C(CH₃)═CH₂), allylidene (e.g., ═CH—CH═CH₂), homoallyl (e.g., —CH₂—CH₂—CH═CH₂), 1-butenyl (—CH═CH—CH₂—CH₃), 2-butenyl (—CH₂—CH═CH—CH₃), 3-methyl-2-butenyl or prenyl (—CH₂—CH═C(CH₃)₂), 3-butenyl (—CH₂—CH₂—CH═CH₂), 4-methyl-3-pentenyl (—CH₂—CH₂—CH═C(CH₃)₂), and the like.

By “alkenylene” is meant a multivalent (e.g., bivalent) form of an alkenyl group, as defined herein. The alkenylene group can be substituted or unsubstituted. For example, the alkenylene group can be substituted with one or more substitution groups, as described herein for alkyl. Exemplary alkenylene groups include, e.g., vinylene (—CH═CH—), vinylidene (e.g., >C═CH₂), ethanediylidene (e.g., ═CH—CH═), ethylidene (e.g., >CH—CH₃), allylidene (e.g., >CH—CH═CH₂), propenylene (e.g., —CH₂—CH═CH— or —CH₂═C═CH—), 1-propanyl-3-ylidene (═CH—CH₂—CH₂—), 2-butenylene (—CH₂—CH═CH—CH₂—), and the like.

By “alkoxy” is meant an —O-Ak group, in which Ak is an alkyl group, as defined herein.

By “alkoxyalkyl” is meant an alkyl group, as defined herein, substituted by an alkoxy group, as defined herein.

By “alkyl” and the prefix “alk” is meant a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic (e.g., C₃₋₂₄ cycloalkyl) or acyclic. The alkyl group can be branched or unbranched. The alkyl group can also be substituted or unsubstituted. For example, the alkyl group can be substituted with one, two, three or, in the case of alkyl groups of two carbons or more, four substituents independently selected from the group consisting of: (1) C₁₋₆ alkoxy (e.g., —O-Ak, wherein Ak is optionally substituted C₁₋₆ alkyl); (2) C₁₋₆ alkylsulfinyl (e.g., —S(O)-Ak, wherein Ak is optionally substituted C₁₋₆ alkyl); (3) C₁₋₆ alkylsulfonyl (e.g., —SO₂-Ak, wherein Ak is optionally substituted C₁₋₆ alkyl); (4) amino (e.g., —NR^{N1}R^{N2}, where each of R^{N1} and R^{N2} is, independently, H or optionally substituted alkyl, or R^{N1} and R^{N2}, taken together with the nitrogen atom to which each are attached, form a heterocyclyl group); (5) aryl; (6) arylalkoxy (e.g., —O-L-Ar, wherein L is a bivalent form of optionally substituted alkyl and Ar is optionally substituted aryl); (7) aryloyl (e.g., —C(O)—Ar, wherein Ar is optionally substituted aryl); (8) azido (e.g., —N₃); (9) cyano (e.g., —CN); (10) carboxyaldehyde (e.g., —C(O)H); (11) C₃₋₈ cycloalkyl (e.g., a monovalent saturated or unsaturated non-aromatic cyclic C₃₋₈ hydrocarbon group); (12) halo (e.g., F, Cl, Br, or I); (13) heterocyclyl (e.g., a 5-, 6- or 7-membered ring, unless otherwise specified, containing one, two, three, or four non-carbon heteroatoms, such as nitrogen, oxygen, phosphorous, sulfur, or halo); (14) heterocyclyloxy (e.g., —O-Het, wherein Het is heterocyclyl, as described herein); (15) heterocyclyloyl (e.g., —C(O)—Het, wherein Het is heterocyclyl, as described herein); (16) hydroxyl (e.g., —OH); (17) N-protected amino; (18) nitro (e.g., —NO₂); (19) oxo (e.g., ═O); (20) C₃₋₈ spirocyclyl (e.g., an alkylene or heteroalkylene diradical, both ends of which are bonded to the same carbon atom of the parent group); (21) C₁₋₆ thioalkoxy (e.g., —S-Ak, wherein Ak is optionally substituted C₁₋₆ alkyl); (22) thiol (e.g., —SH); (23) —CO₂R^{A}, where R^{A} is selected from the group consisting of (a) hydrogen, (b) C₁₋₆ alkyl, (c) C₄₋₁₈ aryl, and (d) (C₄₋₁₈ aryl) C₁₋₆ alkyl (e.g., -L-Ar, wherein L is a bivalent form of optionally substituted alkyl group and Ar is optionally substituted aryl); (24) —C(O)NR^{B}R^{C}, where each of RB and R^{C} is, independently, selected from the group consisting of (a) hydrogen, (b) C₁₋₆ alkyl, (c) C₄₋₁₈ aryl, and (d) (C₄₋₁₈ aryl) C₁₋₆ alkyl (e.g., -L-Ar, wherein L is a bivalent form of optionally substituted alkyl group and Ar is optionally substituted aryl); (25) —SO₂R^{D}, where R^{D} is selected from the group consisting of (a) C₁₋₆ alkyl, (b) C₄₋₁₈ aryl, and (c) (C₄₋₁₈ aryl) C₁₋₆ alkyl (e.g., -L-Ar, wherein L is a bivalent form of optionally substituted alkyl group and Ar is optionally substituted aryl); (26) —SO₂NR^{E}R^{F}, where each of R^{E} and R^{F} is, independently, selected from the group consisting of (a) hydrogen, (b) C₁₋₆ alkyl, (c) C₄₋₁₈ aryl, and (d) (C₄₋₁₈ aryl) C₁₋₆ alkyl (e.g., -L-Ar, wherein L is a bivalent form of optionally substituted alkyl group and Ar is optionally substituted aryl); and (27) —NR^{G}R^{H}, where each of R^{G} and R^{H} is, independently, selected from the group consisting of (a) hydrogen, (b) an N-protecting group, (c) C₁₋₆ alkyl, (d) C₂₋₆ alkenyl (e.g., optionally substituted alkyl having one or more double bonds), (e) C₂₋₆ alkynyl (e.g., optionally substituted alkyl having one or more triple bonds), (f) C₄₋₁₈ aryl, (g) (C₄₋₁₈ aryl) C₁₋₆ alkyl (e.g., L-Ar, wherein L is a bivalent form of optionally substituted alkyl group and Ar is optionally substituted aryl), (h) C₃₋₈ cycloalkyl, and (i) (C₃₋₈ cycloalkyl) C₁₋₆ alkyl (e.g., -L-Cy, wherein L is a bivalent form of optionally substituted alkyl group and Cy is optionally substituted cycloalkyl, as described herein), wherein in one embodiment no two groups are bound to the nitrogen atom through a carbonyl group or a sulfonyl group. The alkyl group can be a primary, secondary, or tertiary alkyl group substituted with one or more substituents (e.g., one or more halo or alkoxy).

In some embodiments, the unsubstituted alkyl group is a C₁₋₃, C₁₋₆, C₁₋₁₂, C₁₋₁₆, C₁₋₁₈, C₁₋₂₀, C₁₋₂₄, C₂₋₃, C₂₋₆, C₂₋₁₂, C₂₋₁₆, C₂₋₁₈, C₂₋₂₀, C₂₋₂₄, C₃₋₆, C₃₋₁₂, C₃₋₁₆, C₃₋₁₈, C₃₋₂₀, C₃₋₂₄, C₄₋₆, C₄₋₁₂, C₄₋₁₆, C₄₋₁₈, C₄₋₂₀, C₄₋₂₄, C₅₋₆, C₅₋₁₂, C₅₋₁₆, C₅₋₁₈, C₅₋₂₀, C₅₋₂₄, C₆₋₁₂, C₆₋₁₆, C₆₋₁₈, C₆₋₂₀, C₆₋₂₄, C₇₋₁₂, C₇₋₁₆, C₇₋₁₈, C₇₋₂₀, C₇₋₂₄, C₈₋₁₂, C₈₋₁₆, C₈₋₁₈, C₈₋₂₀, C₈₋₂₄, C₉₋₁₂, C₉₋₁₆, C₉₋₁₈, C₉₋₂₀, C₉₋₂₄, C₁₀₋₁₂, C₁₀₋₁₆, C₁₀₋₁₈, C₁₀₋₂₀, or C₁₋₂₄ alkyl group.

By “alkylene” is meant a multivalent (e.g., bivalent) form of an alkyl group, as described herein. Exemplary alkylene groups include methylene, ethylene, propylene, butylene, etc. In some embodiments, the alkylene group is a C₁₋₃, C₁₋₆, C₁₋₁₂, C₁₋₁₆, C₁₋₁₈, C₁₋₂₀, C₁₋₂₄, C₂₋₃, C₂₋₆, C₂₋₁₂, C₂₋₁₆, C₂₋₁₈, C₂₋₂₀, or C₂₋₂₄ alkylene group. The alkylene group can be branched or unbranched. The alkylene group can also be substituted or unsubstituted. For example, the alkylene group can be substituted with one or more substitution groups, as described herein for alkyl. Exemplary alkylene groups include, e.g., methylene (e.g., ═CH₂, >CH₂, or —CH₂—), ethylene (—CH₂—CH₂—), propylene (e.g., —CH(CH₃)—CH₂— or —CH₂—CH₂—CH₂—), and the like.

By “alkynyl” is meant an optionally substituted C₂₋₂₄ alkyl group having one or more triple bonds. The alkynyl group can be cyclic or acyclic and is exemplified by ethynyl, 1-propynyl, and the like. The alkynyl group can also be substituted or unsubstituted. For example, the alkynyl group can be substituted with one or more substitution groups, as described herein for alkyl.

By “amide bond” is meant —C(O)NR^{N1}— or —NR^{N1}C(O)—, where R^{N1} is H or optionally substituted alkyl. A non-limiting amide bond includes —C(O)NH—.

By “amido” is meant —C(O)NR^{N1}R^{N2}, where each of R^{N1} and R^{N2} is, independently, H or optionally substituted alkyl, or R^{N1} and R^{N2}, taken together with the nitrogen atom to which each are attached, form a heterocyclyl group, as defined herein.

By “amidoalkyl” is meant an alkyl group, as defined herein, substituted by an amido group, as defined herein.

By “amino” is meant —NR^{N1}R^{N2}, where each of R^{N1} and R^{N2} is, independently, H, optionally substituted alkyl, or optionally substituted aryl, or R^{N1} and R^{N2}, taken together with the nitrogen atom to which each are attached, form a heterocyclyl group, as defined herein.

By “aminoalkyl” is meant an alkyl group, as defined herein, substituted by an amino group, as defined herein.

By “aminoaryl” is meant an aryl group, as defined herein, substituted by an amino group, as defined herein.

By “aralkyl” or “arylalkyl” is meant -Ak-Ar, in which Ak is an optionally substituted alkylene, as defined herein, and Ar is an optionally substituted aryl, as defined herein. The aralkyl group can be substituted or unsubstituted. For example, the aralkyl group can be substituted with one or more substitution groups, as described herein for aryl and/or alkyl. Non-limiting unsubstituted aralkyl groups are of from 7 to 16 carbons (C₇₋₁₆ aralkyl), as well as those having an aryl group with 4 to 18 carbons and an alkylene group with 1 to 6 carbons (i.e., —C₁₋₆ alkylene-C₄₋₁₈ aryl).

By “aryl” is meant a group that contains any carbon-based aromatic group including, but not limited to, phenyl, benzyl, anthracenyl, anthryl, benzocyclobutenyl, benzocyclooctenyl, biphenylyl, chrysenyl, dihydroindenyl, fluoranthenyl, indacenyl, indenyl, naphthyl, phenanthryl, phenoxybenzyl, picenyl, pyrenyl, terphenyl, and the like, including fused benzo-C₄₋₈ cycloalkyl radicals (e.g., as defined herein) such as, for instance, indanyl, tetrahydronaphthyl, fluorenyl, and the like. The term aryl also includes heteroaryl, which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term non-heteroaryl, which is also included in the term aryl, defines a group that contains an aromatic group that does not contain a heteroatom. The aryl group can be substituted or unsubstituted. The aryl group can be substituted with one, two, three, four, or five substituents independently selected from the group consisting of: (1) C₁₋₆ alkanoyl (e.g., —C(O)-Ak, wherein Ak is optionally substituted C₁₋₆ alkyl); (2) C₁₋₆ alkyl; (3) C₁₋₆ alkoxy (e.g., —O-Ak, wherein Ak is optionally substituted C₁₋₆ alkyl); (4) C₁₋₆ alkoxy-C₁₋₆ alkyl (e.g., -L-O-Ak, wherein L is a bivalent form of optionally substituted alkyl group and Ak is optionally substituted C₁₋₆ alkyl); (5) C₁₋₆ alkylsulfinyl (e.g., —S(O)-Ak, wherein Ak is optionally substituted C₁₋₆ alkyl); (6) C₁₋₆ alkylsulfinyl-C₁₋₆ alkyl (e.g., -L-S(O)-Ak, wherein L is a bivalent form of optionally substituted alkyl group and Ak is optionally substituted C₁₋₆ alkyl); (7) C₁₋₆ alkylsulfonyl (e.g., —SO₂-Ak, wherein Ak is optionally substituted C₁₋₆ alkyl); (8) C₁₋₆ alkylsulfonyl-C₁₋₆ alkyl (e.g., -L-SO₂-Ak, wherein L is a bivalent form of optionally substituted alkyl group and Ak is optionally substituted C₁₋₆ alkyl); (9) aryl; (10) amino (e.g., —NR^{N1}R^{N2} where each of R^{N1} and R^{N2} is, independently, H or optionally substituted alkyl, or R^{N1} and R^{N2} taken together with the nitrogen atom to which each are attached, form a heterocyclyl group); (11) C₁₋₆ aminoalkyl (e.g., an alkyl group, as defined herein, substituted by one or more —NR^{N1}R^{N2} groups, as described herein); (12) heteroaryl (e.g., a subset of heterocyclyl groups (e.g., a 5-, 6- or 7-membered ring, unless otherwise specified, containing one, two, three, or four non-carbon heteroatoms), which are aromatic); (13) (C₄₋₁₈ aryl) C₁₋₆ alkyl (e.g., -L-Ar, wherein L is a bivalent form of optionally substituted alkyl and Ar is optionally substituted aryl); (14) aryloyl (e.g., —C(O)—Ar, wherein Ar is optionally substituted aryl); (15) azido (e.g., —N₃); (16) cyano (e.g., —CN); (17) C₁₋₆ azidoalkyl (e.g., an alkyl group, as defined herein, substituted by one or more azido groups, as described herein); (18) carboxyaldehyde (e.g., —C(O)H); (19) carboxyaldehyde-C₁₋₆ alkyl (e.g., an alkyl group, as defined herein, substituted by one or more carboxyaldehyde groups, as described herein); (20) C₃₋₈ cycloalkyl (e.g., a monovalent saturated or unsaturated non-aromatic cyclic C₃₋₈ hydrocarbon group); (21) (C₃₋₈ cycloalkyl) C₁₋₆ alkyl (e.g., an alkyl group, as defined herein, substituted by one or more cycloalkyl groups, as described herein); (22) halo (e.g., F, Cl, Br, or I); (23) C₁₋₆ haloalkyl (e.g., an alkyl group, as defined herein, substituted by one or more halo groups, as described herein); (24) heterocyclyl (e.g., a 5-, 6- or 7-membered ring, unless otherwise specified, containing one, two, three, or four non-carbon heteroatoms, such as nitrogen, oxygen, phosphorous, sulfur, or halo); (25) heterocyclyloxy (e.g., —O-Het, wherein Het is heterocyclyl, as described herein); (26) heterocyclyloyl (e.g., —C(O)—Het, wherein Het is heterocyclyl, as described herein); (27) hydroxyl (e.g., —OH); (28) C₁₋₆ hydroxyalkyl (e.g., an alkyl group, as defined herein, substituted by one or more hydroxyl, as described herein); (29) nitro (e.g., —NO₂); (30) C₁₋₆ nitroalkyl (e.g., an alkyl group, as defined herein, substituted by one or more nitro, as described herein); (31) N-protected amino; (32) N-protected amino-C₁₋₆ alkyl (e.g., an alkyl group, as defined herein, substituted by one or more N-protected amino groups); (33) oxo (e.g., ═O); (34) C₁₋₆ thioalkoxy (e.g., —S-Ak, wherein Ak is optionally substituted C₁₋₆ alkyl); (35) thio-C₁₋₆ alkoxy-C₁₋₆ alkyl (e.g., -L-S-Ak, wherein L is a bivalent form of optionally substituted alkyl and Ak is optionally substituted C₁₋₆ alkyl); (36) —(CH₂)ᵣCO₂R^{A}, where r is an integer of from zero to four, and R^{A} is selected from the group consisting of (a) hydrogen, (b) C₁₋₆ alkyl, (c) C₄₋₁₈ aryl, and (d) (C₄₋₁₈ aryl) C₁₋₆ alkyl (e.g., -L-Ar, wherein L is a bivalent form of optionally substituted alkyl and Ar is optionally substituted aryl); (37) —(CH₂)ᵣCONR^{B}R^{C}, where r is an integer of from zero to four and where each R^{B} and R^{C} is independently selected from the group consisting of (a) hydrogen, (b) C₁₋₆ alkyl, (c) C₄₋₁₈ aryl, and (d) (C₄₋₁₈ aryl) C₁₋₆ alkyl (e.g., -L-Ar, wherein L is a bivalent form of optionally substituted alkyl and Ar is optionally substituted aryl); (38) —(CH₂)ᵣSO₂R^{D}, where r is an integer of from zero to four and where R^{D} is selected from the group consisting of (a) C₁₋₆ alkyl, (b) C₄₋₁₈ aryl, and (c) (C₄₋₁₈ aryl) C₁₋₆ alkyl (e.g., -L-Ar, wherein L is a bivalent form of optionally substituted alkyl and Ar is optionally substituted aryl); (39) —(CH₂)ᵣSO₂NR^{E}R^{F}, where r is an integer of from zero to four and where each of R^{E} and R^{F} is, independently, selected from the group consisting of (a) hydrogen, (b) C₁₋₆ alkyl, (c) C_{4_18} aryl, and (d) (C₄₋₁₈ aryl) C₁₋₆ alkyl (e.g., -L-Ar, wherein L is a bivalent form of optionally substituted alkyl and Ar is optionally substituted aryl); (40) —(CH₂)ᵣNR^{G}R^{H}, where r is an integer of from zero to four and where each of R^{G} and R^{H} is, independently, selected from the group consisting of (a) hydrogen, (b) an N-protecting group, (c) C₁₋₆ alkyl, (d) C₂₋₆ alkenyl (e.g., optionally substituted alkyl having one or more double bonds), (e) C₂₋₆ alkynyl (e.g., optionally substituted alkyl having one or more triple bonds), (f) C₄₋₁₈ aryl, (g) (C₄₋₁₈ aryl) C₁₋₆ alkyl (e.g., -L-Ar, wherein L is a bivalent form of optionally substituted alkyl and Ar is optionally substituted aryl), (h) C₃₋₈ cycloalkyl, and (i) (C₃₋₈ cycloalkyl) C₁₋₆ alkyl (e.g., -L-Cy, wherein L is a bivalent form of optionally substituted alkyl and Cy is optionally substituted cycloalkyl, as described herein), wherein in one embodiment no two groups are bound to the nitrogen atom through a carbonyl group or a sulfonyl group; (41) thiol (e.g., —SH); (42) perfluoroalkyl (e.g., an alkyl group having each hydrogen atom substituted with a fluorine atom); (43) perfluoroalkoxy (e.g., —OR^{f}, where R^{f} is an alkyl group having each hydrogen atom substituted with a fluorine atom); (44) aryloxy (e.g., —OAr, where Ar is optionally substituted aryl); (45) cycloalkoxy (e.g., —O-Cy, wherein Cy is optionally substituted cycloalkyl, as described herein); (46) cycloalkylalkoxy (e.g., —O-L-Cy, wherein L is a bivalent form of optionally substituted alkyl and Cy is optionally substituted cycloalkyl, as described herein); and (47) arylalkoxy (e.g., —O-L-Ar, wherein L is a bivalent form of optionally substituted alkyl and Ar is optionally substituted aryl). In particular embodiments, an unsubstituted aryl group is a C₄₋₁₈, C₄₋₁₄, C₄₋₁₂, C₄₋₁₀, C₆₋₁₈, C₆₋₁₄, C₆₋₁₂, or C₆₋₁₀ aryl group.

By “(aryl)(alkyl)ene” is meant a bivalent form including an arylene group, as described herein, attached to an alkylene or a heteroalkylene group, as described herein. In some embodiments, the (aryl)(alkyl)ene group is -L-Ar— or -L-Ar-L- or —Ar-L-, in which Ar is an arylene group and each L is, independently, an optionally substituted alkylene group or an optionally substituted heteroalkylene group.

By “arylene” is meant a multivalent (e.g., bivalent) form of an aryl group, as described herein. Non-limiting arylene groups include phenylene (e.g., such as benzene-1,4-diyl, benzene-1,3,4-triyl, or benzene-1,2,4,5-tetrayl), naphthylene (e.g., such as naphthalene-2,6-diyl, naphthalene-2,7-diyl, naphthalene-2,3-diyl, naphthalene-1,5-diyl, or naphthalene-2,3,6,7-tetrayl), biphenylene, triphenylene, diphenyl ether, acenaphthenylene, anthrylene (e.g., such as anthracene-2,6-diyl, anthracene-2,7-diyl, or anthracene-2,3,6,7-tetrayl), or phenanthrylene (e.g., such as phenanthrene-2,7-diyl, phenanthrene-1,8-diyl, or phenanthrene-1,8,9,10-tetrayl). In some embodiments, the arylene group is a C₄₋₁₈, C₄₋₁₄, C₄₋₁₂, C₄₋₁₀, C₆₋₁₈, C₆₋₁₄, C₆₋₁₂, or C₆₋₁₀ arylene group. The arylene group can be branched or unbranched. The arylene group can also be substituted or unsubstituted. For example, the arylene group can be substituted with one or more substitution groups, as described herein for aryl. Arylene groups can be divalent, trivalent, or tetravalent forms of any herein.

By “carbonyl” is meant a —C(O)— group, which can also be represented as >C═O.

By “carboxyl” is meant a —CO₂H group.

By “carboxyalkyl” is meant an alkyl group, as described herein, substituted with one or more —CO₂H groups.

By “carboxyaryl” is meant an aryl group, as described herein, substituted with one or more —CO₂H groups.

By “cyclic anhydride” is meant a 3-, 4-, 5-, 6- or 7-membered ring (e.g., a 5-, 6- or 7-membered ring), unless otherwise specified, having a —C(O)—O—C(O)— group within the ring. The term “cyclic anhydride” also includes bicyclic, tricyclic and tetracyclic groups in which any of the above rings is fused to one, two, or three rings independently selected from the group consisting of an aryl ring, a cyclohexane ring, a cyclohexene ring, a cyclopentane ring, a cyclopentene ring, and another monocyclic heterocyclic ring. Exemplary cyclic anhydride groups include a radical formed from succinic anhydride, glutaric anhydride, maleic anhydride, phthalic anhydride, isochroman-1,3-dione, oxepanedione, tetrahydrophthalic anhydride, hexahydrophthalic anhydride, pyromellitic dianhydride, naphthalic anhydride, 1,2-cyclohexanedicarboxylic anhydride, etc., by removing one or more hydrogen. Other exemplary cyclic anhydride groups include dioxotetrahydrofuranyl, dioxodihydroisobenzofuranyl, etc. The cyclic anhydride group can also be substituted or unsubstituted. For example, the cyclic anhydride group can be substituted with one or more groups including those described herein for heterocyclyl.

By “cyclic imide” is meant a 3-, 4-, 5-, 6- or 7-membered ring (e.g., a 5-, 6- or 7-membered ring), unless otherwise specified, having a —C(O)—NR^{N1}—C(O)— group within the ring, where R^{N1} is H, optionally substituted alkyl, or optionally substituted aryl, as defined herein. The term “cyclic imide” also includes bicyclic, tricyclic and tetracyclic groups in which any of the above rings is fused to one, two, or three rings independently selected from the group consisting of an aryl ring, a cyclohexane ring, a cyclohexene ring, a cyclopentane ring, a cyclopentene ring, and another monocyclic heterocyclic ring. Exemplary cyclic imide groups include a radical formed from succinimide, glutaric imide, maleimide, phthalimide, tetrahydrophthalimide, hexahydrophthalimide, pyromellitic diimide, naphthalimide, etc., by removing one or more hydrogen. Other exemplary cyclic imide groups include succinimido, phthalimido, etc. The cyclic imide group can also be substituted or unsubstituted. For example, the cyclic imide group can be substituted with one or more groups including those described herein for heterocyclyl.

By “cycloalkyl” is meant a monovalent saturated or unsaturated non-aromatic cyclic hydrocarbon group of from three to eight carbons, unless otherwise specified, and is exemplified by cyclopropyl, cyclobutyl, cyclopentyl, cyclohexyl, cycloheptyl, bicyclo[2.2.1.]heptyl, and the like. The cycloalkyl group can also be substituted or unsubstituted. For example, the cycloalkyl group can be substituted with one or more groups including those described herein for alkyl. Exemplary cycloalkyl groups include C₃₋₆ cycloalkyl and C₃₋₈ cycloalkyl.

By “epoxy” is meant -L-R^{H}, in which L is a linker and R^{H} is an optionally substituted oxiranyl group. In particular embodiments, L can be a bond, optionally substituted alkylene, or optionally substituted heteroalkylene; and R^{H} can be monovalent, saturated cycloalkyl group having one oxygen atom and two carbon atoms, and in which each carbon atom can include hydrogen atoms or the hydrogen atoms can be substituted with one or more groups including those described herein for alkyl. In other embodiments, R^{H} is an optionally substituted 2-oxiranyl, in which each H in

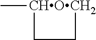

can be substituted with one or more substituents described herein for alkyl.

By “ester bond” is meant is meant —C(O)O— or —OC(O)—.

By “halo” is meant F, Cl, Br, or I.

By “haloalkyl” is meant an alkyl group, as defined herein, substituted with one or more halo.

By “heteroalkylene” is meant a bivalent form of an alkylene group, as defined herein, containing one, two, three, or four non-carbon heteroatoms (e.g., independently selected from the group consisting of nitrogen, oxygen, phosphorous, sulfur, selenium, or halo). The heteroalkylene group can be substituted or unsubstituted. For example, the heteroalkylene group can be substituted with one or more substitution groups, as described herein for alkyl. Non-limiting heteroalkylene groups include, e.g., —O-Ak-, -Ak-O—, —S-Ak-, or -Ak-S—, in which Ak is an optionally substituted alkylene, as described herein.

By “(hetero)cycloalkyl” is meant a saturated cycloalkyl group, as defined herein, having one or more carbon atoms may be replaced by a non-carbon heteroatom (e.g., N, O or S). Examples of (hetero)cycloalkyl are oxiranyl (e.g., 2-oxiranyl, such as

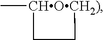

oxetanyl (e.g., 3-oxetanyl, such as

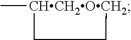

or 2-oxetanyl

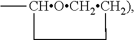

and tetrahydrofuryl (e.g., 2-tetrahydrofuryl or 3-tetrahydrofuryl). The (hetero)cycloalkyl group can also be substituted or unsubstituted. For example, the (hetero)cycloalkyl group can be substituted with one or more groups including those described herein for alkyl. Exemplary (hetero)cycloalkyl groups include C₂₋₆ cycloalkyl and C₂₋₈ cycloalkyl. Yet other exemplary (hetero)cycloalkyl groups include a cyclic ether group, in which the non-carbon heteroatom is O.

By “heterocyclyl” is meant a 3-, 4-, 5-, 6- or 7-membered ring (e.g., a 5-, 6-, or 7-membered ring), unless otherwise specified, containing one, two, three, or four non-carbon heteroatoms (e.g., independently selected from the group consisting of nitrogen, oxygen, phosphorous, sulfur, selenium, or halo). The 3-membered ring has zero to one double bonds, the 4- and 5-membered ring has zero to two double bonds, and the 6- and 7-membered rings have zero to three double bonds. The term “heterocyclyl” also includes bicyclic, tricyclic and tetracyclic groups in which any of the above heterocyclic rings is fused to one, two, or three rings independently selected from the group consisting of an aryl ring, a cyclohexane ring, a cyclohexene ring, a cyclopentane ring, a cyclopentene ring, and another monocyclic heterocyclic ring, such as indolyl, quinolyl, isoquinolyl, tetrahydroquinolyl, benzofuryl, benzothienyl and the like. Heterocyclics include acridinyl, adenyl, alloxazinyl, azaadamantanyl, azabenzimidazolyl, azabicyclononyl, azacycloheptyl, azacyclooctyl, azacyclononyl, azahypoxanthinyl, azaindazolyl, azaindolyl, azecinyl, azepanyl, azepinyl, azetidinyl, azetyl, aziridinyl, azirinyl, azocanyl, azocinyl, azonanyl, benzimidazolyl, benzisothiazolyl, benzisoxazolyl, benzodiazepinyl, benzodiazocinyl, benzodihydrofuryl, benzodioxepinyl, benzodioxinyl, benzodioxanyl, benzodioxocinyl, benzodioxolyl, benzodithiepinyl, benzodithiinyl, benzodioxocinyl, benzofuranyl, benzophenazinyl, benzopyranonyl, benzopyranyl, benzopyrenyl, benzopyronyl, benzoquinolinyl, benzoquinolizinyl, benzothiadiazepinyl, benzothiadiazolyl, benzothiazepinyl, benzothiazocinyl, benzothiazolyl, benzothienyl, benzothiophenyl, benzothiazinonyl, benzothiazinyl, benzothiopyranyl, benzothiopyronyl, benzotriazepinyl, benzotriazinonyl, benzotriazinyl, benzotriazolyl, benzoxathiinyl, benzotrioxepinyl, benzoxadiazepinyl, benzoxathiazepinyl, benzoxathiepinyl, benzoxathiocinyl, benzoxazepinyl, benzoxazinyl, benzoxazocinyl, benzoxazolinonyl, benzoxazolinyl, benzoxazolyl, benzylsultamyl benzylsultimyl, bipyrazinyl, bipyridinyl, carbazolyl (e.g., 4H-carbazolyl), carbolinyl (e.g., (3-carbolinyl), chromanonyl, chromanyl, chromenyl, cinnolinyl, coumarinyl, cytdinyl, cytosinyl, decahydroisoquinolinyl, decahydroquinolinyl, diazabicyclooctyl, diazetyl, diaziridinethionyl, diaziridinonyl, diaziridinyl, diazirinyl, dibenzisoquinolinyl, dibenzoacridinyl, dibenzocarbazolyl, dibenzofuranyl, dibenzophenazinyl, dibenzopyranonyl, dibenzopyronyl (xanthonyl), dibenzoquinoxalinyl, dibenzothiazepinyl, dibenzothiepinyl, dibenzothiophenyl, dibenzoxepinyl, dihydroazepinyl, dihydroazetyl, dihydrofuranyl, dihydrofuryl, dihydroisoquinolinyl, dihydropyranyl, dihydropyridinyl, dihydroypyridyl, dihydroquinolinyl, dihydrothienyl, dihydroindolyl, dioxanyl, dioxazinyl, dioxindolyl, dioxiranyl, dioxenyl, dioxinyl, dioxobenzofuranyl, dioxolyl, dioxotetrahydrofuranyl, dioxothiomorpholinyl, dithianyl, dithiazolyl, dithienyl, dithiinyl, furanyl, furazanyl, furoyl, furyl, guaninyl, homopiperazinyl, homopiperidinyl, hypoxanthinyl, hydantoinyl, imidazolidinyl, imidazolinyl, imidazolyl, indazolyl (e.g., 1H-indazolyl), indolenyl, indolinyl, indolizinyl, indolyl (e.g., 1H-indolyl or 3H-indolyl), isatinyl, isatyl, isobenzofuranyl, isochromanyl, isochromenyl, isoindazoyl, isoindolinyl, isoindolyl, isopyrazolonyl, isopyrazolyl, isoxazolidiniyl, isoxazolyl, isoquinolinyl, isoquinolinyl, isothiazolidinyl, isothiazolyl, morpholinyl, naphthindazolyl, naphthindolyl, naphthiridinyl, naphthopyranyl, naphthothiazolyl, naphthothioxolyl, naphthotriazolyl, naphthoxindolyl, naphthyridinyl, octahydroisoquinolinyl, oxabicycloheptyl, oxauracil, oxadiazolyl, oxazinyl, oxaziridinyl, oxazolidinyl, oxazolidonyl, oxazolinyl, oxazolonyl, oxazolyl, oxepanyl, oxetanonyl, oxetanyl, oxetyl, oxtenayl, oxindolyl, oxiranyl, oxobenzoisothiazolyl, oxochromenyl, oxoisoquinolinyl, oxoquinolinyl, oxothiolanyl, phenanthridinyl, phenanthrolinyl, phenazinyl, phenothiazinyl, phenothienyl (benzothiofuranyl), phenoxathiinyl, phenoxazinyl, phthalazinyl, phthalazonyl, phthalidyl, phthalimidinyl, piperazinyl, piperidinyl, piperidonyl (e.g., 4-piperidonyl), pteridinyl, purinyl, pyranyl, pyrazinyl, pyrazolidinyl, pyrazolinyl, pyrazolopyrimidinyl, pyrazolyl, pyridazinyl, pyridinyl, pyridopyrazinyl, pyridopyrimidinyl, pyridyl, pyrimidinyl, pyrimidyl, pyronyl, pyrrolidinyl, pyrrolidonyl (e.g., 2-pyrrolidonyl), pyrrolinyl, pyrrolizidinyl, pyrrolyl (e.g., 2H-pyrrolyl), quinazolinyl, quinolinyl, quinolizinyl (e.g., 4H-quinolizinyl), quinoxalinyl, quinuclidinyl, selenazinyl, selenazolyl, selenophenyl, succinimidyl, sulfolanyl, tetrahydrofuranyl, tetrahydrofuryl, tetrahydroisoquinolinyl, tetrahydroisoquinolyl, tetrahydropyridinyl, tetrahydropyridyl (piperidyl), tetrahydropyranyl, tetrahydropyronyl, tetrahydroquinolinyl, tetrahydroquinolyl, tetrahydrothienyl, tetrahydrothiophenyl, tetrazinyl, tetrazolyl, thiadiazinyl (e.g., 6H-1,2,5-thiadiazinyl or 2H,6H-1,5,2-dithiazinyl), thiadiazolyl, thianthrenyl, thianyl, thianaphthenyl, thiazepinyl, thiazinyl, thiazolidinedionyl, thiazolidinyl, thiazolyl, thienyl, thiepanyl, thiepinyl, thietanyl, thietyl, thiiranyl, thiocanyl, thiochromanonyl, thiochromanyl, thiochromenyl, thiodiazinyl, thiodiazolyl, thioindoxyl, thiomorpholinyl, thiophenyl, thiopyranyl, thiopyronyl, thiotriazolyl, thiourazolyl, thioxanyl, thioxolyl, thymidinyl, thyminyl, triazinyl, triazolyl, trithianyl, urazinyl, urazolyl, uretidinyl, uretinyl, uricyl, uridinyl, xanthenyl, xanthinyl, xanthionyl, and the like, as well as modified forms thereof (e.g., including one or more oxo and/or amino), and salts thereof. The heterocyclyl group can be substituted or unsubstituted. For example, the heterocyclyl group can be substituted with one or more substitution groups, as described herein for aryl.

By “(heterocyclyl)(alkyl)ene” is meant a bivalent form including a heterocyclyldiyl group, as described herein, attached to an alkylene or a heteroalkylene group, as described herein. In some embodiments, the (heterocyclyl)(alkyl)ene group is -L-Het-, -L-Het-L-, or -Het-L-, in which Het is a heterocyclyldiyl group and L is an optionally substituted alkylene group or an optionally substituted heteroalkylene group.

By “heterocyclyldiyl” is meant a bivalent form of a heterocyclyl group, as described herein. In one instance, the heterocyclyldiyl is formed by removing a hydrogen from a heterocyclyl group. Exemplary heterocyclyldiyl groups include piperdylidene, quinolinediyl, etc. The heterocyclyldiyl group can also be substituted or unsubstituted. For example, the heterocyclyldiyl group can be substituted with one or more substitution groups, as described herein for heterocyclyl.

By “hydroxyl” is meant —OH.

By “hydroxyalkyl” is meant an alkyl group, as defined herein, substituted by one to three hydroxyl groups, with the proviso that no more than one hydroxyl group may be attached to a single carbon atom of the alkyl group and is exemplified by hydroxymethyl, dihydroxypropyl, and the like.

By “hydroxyaryl” is meant an aryl group, as defined herein, substituted by one to three hydroxyl groups, with the proviso that no more than one hydroxyl group may be attached to a single carbon atom of the aryl group and is exemplified by hydroxyphenyl, dihydroxyphenyl, and the like.

By “oxy” is meant —O—.

By “protecting group” is meant any group intended to protect a reactive group against undesirable synthetic reactions. Commonly used protecting groups are disclosed in “Greene's Protective Groups in Organic Synthesis,” John Wiley & Sons, New York, 2007 (4th ed., eds. P. G. M. Wuts and T. W. Greene), which is incorporated herein by reference. O-protecting groups include an optionally substituted alkyl group (e.g., forming an ether with reactive group O), such as methyl, methoxymethyl, methylthiomethyl, benzoyloxymethyl, t-butoxymethyl, etc.; an optionally substituted alkanoyl group (e.g., forming an ester with the reactive group O), such as formyl, acetyl, chloroacetyl, fluoroacetyl (e.g., perfluoroacetyl), methoxyacetyl, pivaloyl, t-butylacetyl, phenoxyacetyl, etc.; an optionally substituted aryloyl group (e.g., forming an ester with the reactive group O), such as —C(O)—Ar, including benzoyl; an optionally substituted alkylsulfonyl group (e.g., forming an alkylsulfonate with reactive group O), such as —SO₂—R^{S1}, where R^{S1} is optionally substituted C₁₋₁₂ alkyl, such as mesyl or benzylsulfonyl; an optionally substituted arylsulfonyl group (e.g., forming an arylsulfonate with reactive group O), such as —SO₂—R^{S4}, where R^{S4} is optionally substituted C₄₋₁₈ aryl, such as tosyl or phenylsulfonyl; an optionally substituted alkoxycarbonyl or aryloxycarbonyl group (e.g., forming a carbonate with reactive group O), such as —C(O)—OR^{T1}, where R^{T1} is optionally substituted C₁₋₁₂ alkyl or optionally substituted C₄₋₁₈ aryl, such as methoxycarbonyl, methoxymethylcarbonyl, t-butyloxycarbonyl (Boc), or benzyloxycarbonyl (Cbz); or an optionally substituted silyl group (e.g., forming a silyl ether with reactive group O), such as —Si—(R^{T2})₃, where each R^{T2} is, independently, optionally substituted C₁₋₁₂ alkyl or optionally substituted C₄₋₁₈ aryl, such as trimethylsilyl, t-butyldimethylsilyl, or t-butyldiphenylsilyl. N-protecting groups include, e.g., formyl, acetyl, benzoyl, pivaloyl, t-butylacetyl, alanyl, phenylsulfonyl, benzyl, Boc, and Cbz. Such protecting groups can employ any useful agent to cleave the protecting group, thereby restoring the reactivity of the unprotected reactive group.

By “salt” is meant an ionic form of a compound or structure (e.g., any formulas, compounds, or compositions described herein), which includes a cation or anion compound to form an electrically neutral compound or structure. Salts are well known in the art. For example, non-toxic salts are described in Berge S M et al., “Pharmaceutical salts,” J. Pharm. Sci. 1977 January; 66(1):1-19; and in “Handbook of Pharmaceutical Salts: Properties, Selection, and Use,” Wiley-VCH, April 2011 (2nd rev. ed., eds. P. H. Stahl and C. G. Wermuth. The salts can be prepared in situ during the final isolation and purification of the compounds of the invention or separately by reacting the free base group with a suitable organic acid (thereby producing an anionic salt) or by reacting the acid group with a suitable metal or organic salt (thereby producing a cationic salt). Representative anionic salts include acetate, adipate, alginate, ascorbate, aspartate, benzenesulfonate, benzoate, bicarbonate, bisulfate, bitartrate, borate, bromide, butyrate, camphorate, camphorsulfonate, chloride, citrate, cyclopentanepropionate, digluconate, dihydrochloride, diphosphate, dodecylsulfate, edetate, ethanesulfonate, fumarate, glucoheptonate, gluconate, glutamate, glycerophosphate, hemisulfate, heptonate, hexanoate, hydrobromide, hydrochloride, hydroiodide, hydroxyethanesulfonate, hydroxynaphthoate, iodide, lactate, lactobionate, laurate, lauryl sulfate, malate, maleate, malonate, mandelate, mesylate, methanesulfonate, methylbromide, methylnitrate, methylsulfate, mucate, 2-naphthalenesulfonate, nicotinate, nitrate, oleate, oxalate, palmitate, pamoate, pectinate, persulfate, 3-phenylpropionate, phosphate, picrate, pivalate, polygalacturonate, propionate, salicylate, stearate, subacetate, succinate, sulfate, tannate, tartrate, theophyllinate, thiocyanate, triethiodide, toluenesulfonate, undecanoate, valerate salts, and the like. Representative cationic salts include metal salts, such as alkali or alkaline earth salts, e.g., barium, calcium (e.g., calcium edetate), lithium, magnesium, potassium, sodium, and the like; other metal salts, such as aluminum, bismuth, iron, and zinc; as well as nontoxic ammonium, quaternary ammonium, and amine cations, including, but not limited to ammonium, tetramethylammonium, tetraethylammonium, methylamine, dimethylamine, trimethylamine, triethylamine, ethylamine, pyridinium, and the like. Other cationic salts include organic salts, such as chloroprocaine, choline, dibenzylethylenediamine, diethanolamine, ethylenediamine, methylglucamine, and procaine. Yet other salts include ammonium, sulfonium, sulfoxonium, phosphonium, iminium, imidazolium, benzimidazolium, amidinium, guanidinium, phosphazinium, phosphazenium, pyridinium, etc., as well as other cationic groups described herein (e.g., optionally substituted isoxazolium, optionally substituted oxazolium, optionally substituted thiazolium, optionally substituted pyrrolium, optionally substituted furanium, optionally substituted thiophenium, optionally substituted imidazolium, optionally substituted pyrazolium, optionally substituted isothiazolium, optionally substituted triazolium, optionally substituted tetrazolium, optionally substituted furazanium, optionally substituted pyridinium, optionally substituted pyrimidinium, optionally substituted pyrazinium, optionally substituted triazinium, optionally substituted tetrazinium, optionally substituted pyridazinium, optionally substituted oxazinium, optionally substituted pyrrolidinium, optionally substituted pyrazolidinium, optionally substituted imidazolinium, optionally substituted isoxazolidinium, optionally substituted oxazolidinium, optionally substituted piperazinium, optionally substituted piperidinium, optionally substituted morpholinium, optionally substituted azepanium, optionally substituted azepinium, optionally substituted indolium, optionally substituted isoindolium, optionally substituted indolizinium, optionally substituted indazolium, optionally substituted benzimidazolium, optionally substituted isoquinolinum, optionally substituted quinolizinium, optionally substituted dehydroquinolizinium, optionally substituted quinolinium, optionally substituted isoindolinium, optionally substituted benzimidazolinium, and optionally substituted purinium).

By “stereoisomer” is meant any of the various stereoisomeric configurations that may exist for a given compound of the present invention and includes geometric isomers. It is understood that a substituent may be attached at a chiral center of a carbon atom. The term “chiral” refers to molecules which have the property of non-superimposability on their mirror image partner, while the term “achiral” refers to molecules which are superimposable on their mirror image partner. Therefore, the disclosure includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereomers” are stereoisomers that have at least two asymmetric atoms, but which are not mirror-images of each other. The absolute stereochemistry can be specified according to the Cahn-Ingold-Prelog R-S system.

By “thio” is meant an —S— group.

By “attaching,” “attachment,” or related word forms is meant any covalent or non-covalent bonding interaction between two components. Non-covalent bonding interactions include, without limitation, hydrogen bonding, ionic interactions, halogen bonding, electrostatic interactions, π bond interactions, hydrophobic interactions, inclusion complexes, clathration, van der Waals interactions, and combinations thereof.

As used herein, the term “solvent” encompasses pure solvents as well as mixtures of different solvents.

As used herein, the term “fermentation” is used herein to refer to a process whereby a microbial cell converts one or more substrate(s) into a desired product by means of one or more biological conversion steps, without the need for any chemical conversion step.

As used herein, the term “about” means+/−10% of any recited value. As used herein, this term modifies any recited value, range of values, or endpoints of one or more ranges.

As used herein, the terms “top,” “bottom,” “upper,” “lower,” “above,” and “below” are used to provide a relative relationship between structures. The use of these terms does not indicate or require that a particular structure must be located at a particular location in the apparatus.

As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

This written description uses examples to disclose the embodiments, including the best mode, and also to enable those of ordinary skill in the art to make and use the invention.

The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

Note that not all of the activities described above in the general description or the examples are required, that a portion of a specific activity may not be required, and that one or more further activities may be performed in addition to those described. The order in which activities are listed is not necessarily the order in which they are performed.

In this specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

Benefits, other advantages, and solutions to problems have been described herein with regard to specific embodiments. However, the benefits, advantages, solutions to problems, and any feature(s) that may cause any benefit, advantage, or solution to occur or become more pronounced are not to be construed as a critical, required, or essential feature of any or all the claims.

After reading the specification, skilled artisans will appreciate that certain features are, for clarity, described herein in the context of separate embodiments, may also be provided in combination in a single embodiment. Conversely, various features that are, for brevity, described in the context of a single embodiment, may also be provided separately or in any subcombination. Further, references to values stated in ranges include each and every value within that range.

### DETAILED DESCRIPTION OF THE PREFERRED EMBODIMENT(S)

The present disclosure relates to compositions derived from bioreachable molecules, such as amino acids or steroids obtained from microbes (e.g., engineered microbes to overexpress desired biomolecules). Such bioreachable amino acids can be reacted to form a cyclic dimer or a cyclic derivative, which can be further chemically functionalized. In addition, steroids can be reacted to form a functionalized steroid. Such functionalized amino acids, cyclic dimers, and steroids can include, e.g., inclusion of one or more reactive moieties, polymerizable moieties, or others. In turn, such cyclic derivatives and/or functionalized steroids can be employed as a monomer, a polymer, or a copolymer.

In one aspect, the cyclic derivative can include a structure having formula (I), (Ia), (Ib), or (Ic):

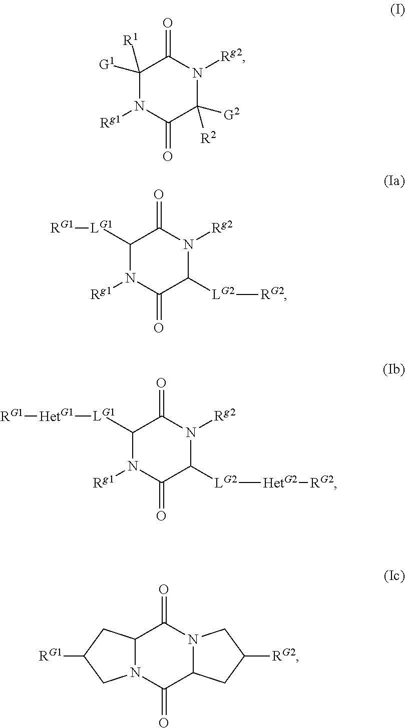

or a salt thereof. In some embodiments, each of G¹, G², R^{G1}, and R^{G2} is or includes, independently, hydroxyl, carboxyl, amino, amido, optionally substituted cyclic anhydride, or optionally substituted cyclic imide. In some embodiments, each of R¹ and R² is, independently, H or optionally substituted alkyl. In yet other embodiments, each of R and R^{g2} is, independently, H, optionally substituted alkyl, optionally substituted aryl, optionally substituted alkaryl, or optionally substituted aralkyl. In particular embodiments, R^{g1} and G¹, taken together with the nitrogen to which R^{g1} is bound; and/or R^{g2} and G², taken together with the nitrogen to which R^{g2} is bound, can optionally form an optionally substituted heterocyclyl.

In some non-limiting embodiments, each of R^{g1} and R^{g2} is not H.

In other non-limiting embodiments, each of L^{G1} and L^{G2} does not comprise aryl or arylene. In other embodiments, at least one of G¹, G², R^{G1}, and R^{G2} does not comprise aryl or arylene. In yet other embodiments, each of G¹, G², R^{G1}, R^{G2} L^{G1}, and L^{G2} does not comprise aryl or arylene.

In another aspect, the functionalized steroid can include a structure having formula (II), (IIa), or (IIb):

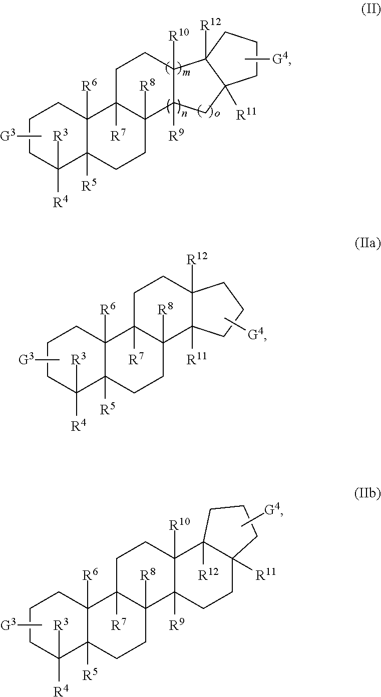

or a salt thereof. In some embodiments, each of G³ and G⁴ is or includes, independently, hydroxyl, carboxyl, amino, amido, optionally substituted cyclic anhydride, or optionally substituted cyclic imide. In particular embodiments, each of R³, R⁴, R⁵, R⁶, R⁷, R⁸, R⁹, R¹⁰, R¹¹, and R¹² is, independently, H or optionally substituted alkyl. In some embodiments, each of m and n is, independently, an integer of from 0 to 1; and o is an integer of from 0 to 2. In other embodiments, each of m, n, and o is 0. In yet other embodiments, each of m, n, and o is 1. In particular embodiments (e.g., in formula (II) or (IIa)), R¹² is optionally substituted alkyl. In other embodiments (e.g., in formula (II) or (IIb)), each of R³, R⁴, R⁶, R⁸, R⁹, and R¹¹ is optionally substituted alkyl.

As can be seen (e.g., in formulas (I), (Ia), (Ib), (Ic), (II), (IIa), or (IIb)), in some instances, G¹, G², G³, and G⁴ can include one or more reactive moieties, which in turn can provide a polymer when the cyclic derivative is employed as a monomer. Within a polymer, the same cyclic derivative can be employed, or two or more different cyclic derivatives may be employed. Illustrative reactive moieties include, e.g., those described herein for R^{G}, R^{G1}, or R^{G2}, such as hydroxyl, optionally substituted hydroxyalkyl, optionally substituted hydroxyaryl, halo, haloalkyl, carboxyl, optionally substituted carboxyalkyl, optionally substituted carboxyaryl, amino, optionally substituted aminoalkyl, optionally substituted aminoaryl, amido, optionally substituted amidoalkyl, optionally substituted alkyl, optionally substituted cyclic anhydride, or optionally substituted cyclic imide.

In some embodiments, one or more of G¹, G², G³, or G⁴ has a structure of -G^{a}OH, in which Ga can be optionally substituted alkylene, optionally substituted arylene, or optionally substituted (aryl)(alkyl)ene. In one embodiment, Ga can be methylene, ethylene, n-propylene, isopropylene, n-butylene, 2-methylpropylene, n-pentylene, 2-methylbutylene, 2,3-dimethylpropylene, 1,4-phenylene, methylene-phenylene, para-methylene-phenylene, ethylene-phenylene, or para-ethylene-phenylene.

In some embodiments, one or more of G¹, G², G³, or G⁴ has a structure of R¹OH, Ar¹OH, R¹NH₂, Ar¹NH₂, R¹R²NH, Ar¹R¹NH, Ar¹Ar²NH,

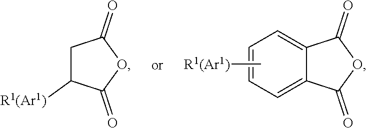

in which each of R¹ and R² includes any alkyl, olefin, or combinations thereof, in bivalent form; and each of Ar¹ and Ar² includes any aromatic ring, such as and not limited to benzene, naphthalene, anthracene, biphenyl, terphenyl, including bivalent forms thereof.

In some embodiments, each of G¹, G², G³, and G⁴ can include one or more linkers (e.g., L^{G}, L^{G1}, L^{G2}, L^{G3}, L^{G4}, Ak^{G1}, Ak^{G3}, Ar^{G1}, Ar^{G2}, Ar^{G3}, Ar^{m}, Het^{G1}, or Het^{G2}) attached to a reactive moiety (e.g., R^{G}, R^{G1}, or R^{G2}). Illustrative linkers include, e.g., a covalent bond, an amide bond, —NR^{N1}— (in which R^{N1} is H or optionally substituted alkyl), an ester bond, oxy, carbonyl, optionally substituted alkylene, optionally substituted alkenylene, optionally substituted heteroalkylene, optionally substituted arylene, optionally substituted (aryl)(alkyl)ene, optionally substituted heterocyclyldiyl, or optionally substituted (heterocyclyl)(alkyl)ene, as well as combinations thereof. In particular embodiments, the linker does not include an aryl or arylene moiety. Yet other linkers can include -L^{G1}-L^{G3}-, -L^{G3}-L^{G1}-, -L^{G1}-Ar^{G1}—, -L^{G1}-Het^{G1}-, -Het^{G1}-L^{G1}-, -L^{G1}-Ar^{G1}-L^{G3}-, -L^{G3}-Ar^{G1}-L^{G1}-, -L^{G2}-L^{G4}-, -L^{G4}-L^{G2}-, -L^{G2}-Ar^{G2}—, -L^{G2}Ar^{G2}-L^{G4}-, -L^{G2}-Het^{G2}-, -L^{G2}-Ar^{G2}-L^{G4}-, -L^{G4}-Het^{G1}-, -Het^{G1}-Ar^{G1}-L^{G1}-, -Het^{G2}-Het^{G1}-Ar^{G1}-L^{G1}-, -Ak^{G3}-L^{G3}-, Ar^{G1}-L^{G1}-, —Ar^{G3}-L^{G3}, Ar^{G1}-L^{G1}-, and —Ar^{m}— for any L^{G1}, L^{G2}, L^{G3}, L^{G4}, Ak^{G1}, Ak^{G3}, Ar^{G1}, Ar^{G2}, Ar^{m}, Het^{G1}, or Het^{G2} described herein.

For any compound herein (e.g., a cyclic derivative or a functionalized steroid), each of G¹, G², G³, and G⁴ can include -L^{G}-R^{G}, in which L^{G} is a linker (e.g., L^{G1} or L^{G2}) attached to a reactive moiety R^{G} (e.g., R^{G1} or R^{G2}). In some embodiments, L (e.g., L^{G1} or L^{G2}) is a bond or optionally substituted alkylene; and R^{G} (e.g., R^{G1} or R^{G2}) is hydroxyl, optionally substituted hydroxyalkyl, optionally substituted hydroxyaryl, carboxyl, optionally substituted carboxyalkyl, optionally substituted carboxyaryl, amino, optionally substituted aminoalkyl, optionally substituted aminoaryl, amido, optionally substituted amidoalkyl, optionally substituted cyclic anhydride, optionally substituted cyclic imide, optionally substituted alkenyl, optionally substituted alkynyl, optionally substituted epoxy, or optionally substituted (hetero)cycloalkyl (e.g., as for G¹ and G² in formula (I)). In particular embodiments, L^{G} (e.g., L^{G1} or L^{G2}) is a bond or optionally substituted alkylene; and R^{G} (e.g., R^{G1} or R^{G2}) is hydroxyl or amino (e.g., as for G³ and G⁴ in formula (II)).

For cyclic derivatives (e.g., as in formulas (I), (Ia), (Ib), or (Ic)), linkers and reactive moieties may be attached to a side chain present in the amino acid employed to form the cyclic dimer. Exemplary side chains can include, e.g., alkyl, amidoalkyl, aminoalkyl, carboxyalkyl, hydroxyalkyl, phenyl, aryl, aralkyl, hydroxyphenyl, hydroxyaryl, hydroxyaralkyl, or heterocyclyl. Accordingly, each of G¹ and G² can include any of such side chains that has been reacted to provide a linker (e.g., L^{G}, L^{G1}, L^{G2}, L^{G3}, L^{G4}, Ak^{G1}, Ak^{G3}, Ar^{G1}, Ar^{G2}, Het^{G1}, or Het^{G2}) attached to a reactive moiety (e.g., R^{G}, R^{G1}, or R^{G2}).

For functionalized steroids (e.g., as in formulas (II), (IIa), or (IIb)), linkers and reactive moieties may be attached to a side chain present in the tetracyclic or pentacyclic structure of the steroid prior to functionalization. Exemplary side chains include, e.g., linear alkyl, branched alkyl, alkenyl, hydroxyl, hydroxyalkyl, etc. Accordingly, each of G³ and G⁴ can include any of such side chains that has been reacted to provide a linker (e.g., L^{G}, L^{G1}, L^{G2}, L^{G3}, L^{G4}, Ak^{G1}, Ak^{G3}, Ar^{G1}, Ar^{G2}, Het^{G1}, or Het^{G2}) attached to a reactive moiety (e.g., R^{G}, R^{G1}, or R^{G2}).

In one embodiment, the reactive moiety is or includes an optionally substituted cyclic anhydride. Thus, G¹, G², G³, G⁴, G^{L1}, G^{L2}, R^{G}, R^{G1}, or R^{G2} can include such a reactive moiety.

In some embodiments, the optionally substituted cyclic anhydride has a structure of:

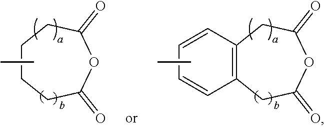

where each of a and b, independently, is an integer of from 0 to 3. In particular embodiments, each of a and b is zero.

In one embodiment, the reactive moiety is or includes an optionally substituted cyclic imide. Thus, G¹, G², G³, G⁴, G^{L}, G, R^{G}, R^{G1}, or R^{G2} can include such a reactive moiety. In some embodiments, the optionally substituted cyclic imide has a structure of:

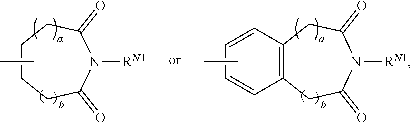

where each of a and b, independently, is an integer of from 0 to 3; and where R^{N1} is H, optionally substituted alkyl, optionally substituted aryl, or optionally substituted aralkyl. In particular embodiments, each of a and b is zero. In other embodiments, R^{N1} is H.

The cyclic derivatives and functionalized steroids can include any useful reactive moiety. In one embodiment, the reactive moiety is or includes hydroxyl, optionally substituted hydroxyalkyl, or optionally substituted hydroxyaryl. In particular embodiments, the cyclic derivative or the functionalized steroid can include a structure selected from the group of:

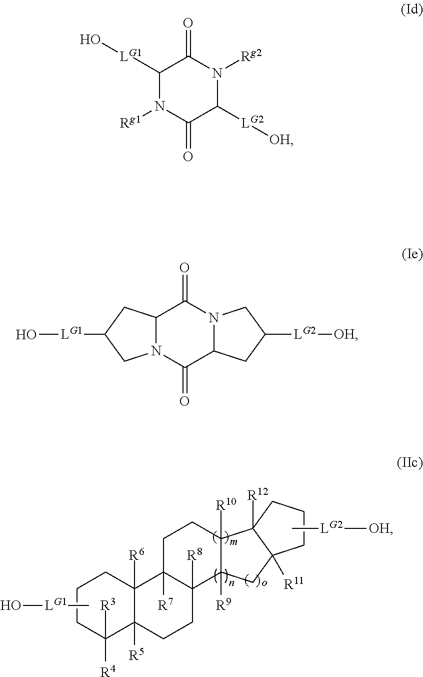

or a salt thereof, in which R^{g1}, R^{g2}, R³, R⁴, R⁵, R⁶, R⁷, R⁸, R⁹, R¹⁰, R¹¹, and R¹² can be any described herein; m, n, and o can be any integer described herein; and L^{G1} and L^{G2} can be any linker described herein. In particular embodiments, each of R^{g1}, R^{g2}, R³, R⁴, R⁵, R⁶, R⁷, R⁸, R⁹, R¹⁰, R¹¹, and R¹² is, independently, H or optionally substituted alkyl. In other embodiments, each of L^{G1} and L^{G2} is, independently, a covalent bond, -Ak-, -hAk-, -Ak-Ar—, -hAk-Ar—, —Ar-Ak-, or —Ar-hAk-, in which Ak is optionally substituted alkylene, hAk is optionally substituted heteroalkylene, and Ar is optionally substituted arylene. In some embodiments, each of L^{G1} and L^{G2} is, independently, a covalent bond, -Ak-, or -hAk-.

In another embodiment, the reactive moiety is or includes amino, optionally substituted aminoalkyl, or optionally substituted aminoaryl. In particular embodiments, the cyclic derivative or the functionalized steroid can include a structure selected from the group of:

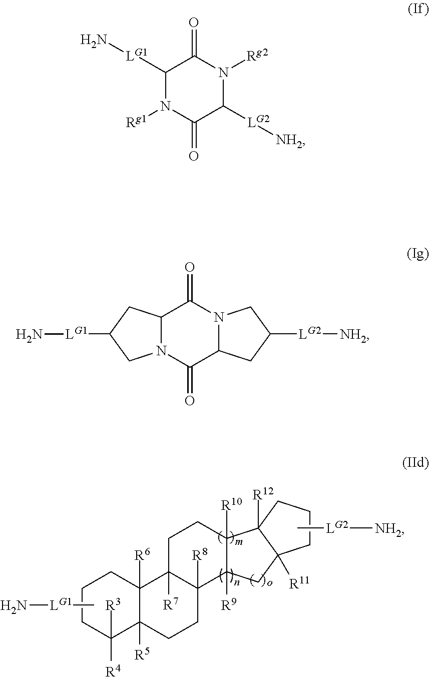

or a salt thereof, in which R^{g1}, R^{g2}, R³, R⁴, R⁵, R⁶, R⁷, R⁸, R⁹, R¹⁰, R¹, and R¹² can be any described herein; m, n, and o can be any integer described herein; and L^{G1} and L^{G2} can be any linker described herein. In particular embodiments, each of R^{g1}, R^{g2}, R³, R⁴, R⁵, R⁶, R⁷, R⁸, R⁹, R¹⁰, R¹¹, and R¹² is, independently, H or optionally substituted alkyl. In other embodiments, each of L^{G1} and L^{G2} is, independently, a covalent bond, -Ak-, -hAk-, -Ak-Ar—, -hAk-Ar—, —Ar-Ak-, or —Ar-hAk-, in which Ak is optionally substituted alkylene, hAk is optionally substituted heteroalkylene, and Ar is optionally substituted arylene. In some embodiments, each of L^{G1} and L^{G2} is, independently, a covalent bond, -Ak-, or -hAk-.

In yet another embodiment, the reactive moiety is or includes optionally substituted cyclic anhydride or optionally substituted cyclic imide. In particular embodiments, the cyclic derivative can include a structure selected from the group of:

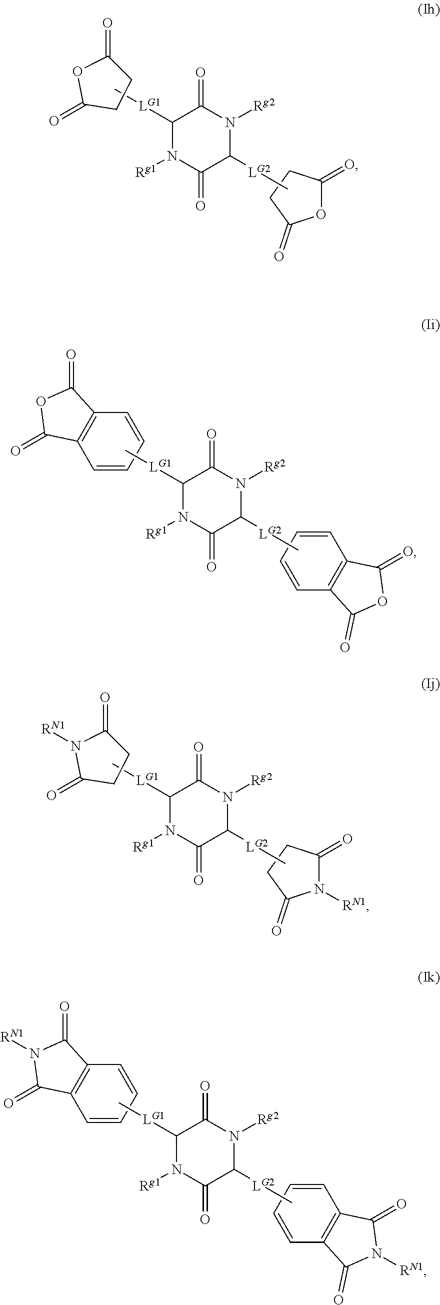

or a salt thereof, in which R^{g1}, R^{g2}, and R^{N1} can be any described herein; and L^{G1} and L^{G2} can be any linker described herein. In particular embodiments, each of R^{g1}, R^{g2}, and R^{N1} is, independently, H or optionally substituted alkyl. In other embodiments, each of L^{G1} and L^{G2} is, independently, a covalent bond, -Ak-, -hAk-, -Ak-Ar—, -hAk-Ar—, —Ar-Ak-, or —Ar-hAk-, in which Ak is optionally substituted alkylene, hAk is optionally substituted heteroalkylene, and Ar is optionally substituted arylene. In some embodiments, each of L^{G1} and L^{G2} is, independently, a covalent bond, -Ak-, or -hAk-.

The compositions herein can be employed as a subunit within a polymer or a copolymer. Exemplary subunits can include a structure having formulas (III), (IIIa), (IV), (IVa), (IVb), (IVc), (V), (Va), (VI), (VIa), (VIb), (VIc), (VII), (VIIa), and (VIII). As can be seen, the subunit can have one or more linkers (e.g., L^{G1}, L^{G2}, L^{G3}, L^{G4}, Ak^{G1}, Ak^{G3}, Ar^{G1}, Ar^{G2}, Ar^{G3}, Ar^{m}, Het^{G1}, Het^{G2}, G^{L1}, and G^{L2}), and the linker within the subunit can include a reaction product arising from reacting two or more reactive moieties (e.g., R^{G}, R^{G1}, or R^{G2}). In one non-limiting embodiment, a first reactive moiety of a first cyclic derivative and a first reactive moiety of a second cyclic derivative can react to form a reaction product of an amide bond or an ester bond. In other instances, the reaction product can provide one or more of the following: hydroxyl, carboxyl, amino, amido, an amide bond, an ester bond, oxy, carbonyl, optionally substituted cyclic anhydride, and optionally substituted cyclic imide.

In some embodiments, the subunit includes one or more heterocyclyl moieties within the linker. In particular embodiments, the subunit includes a structure of formula (III), (IIIa), (V), or (Va):

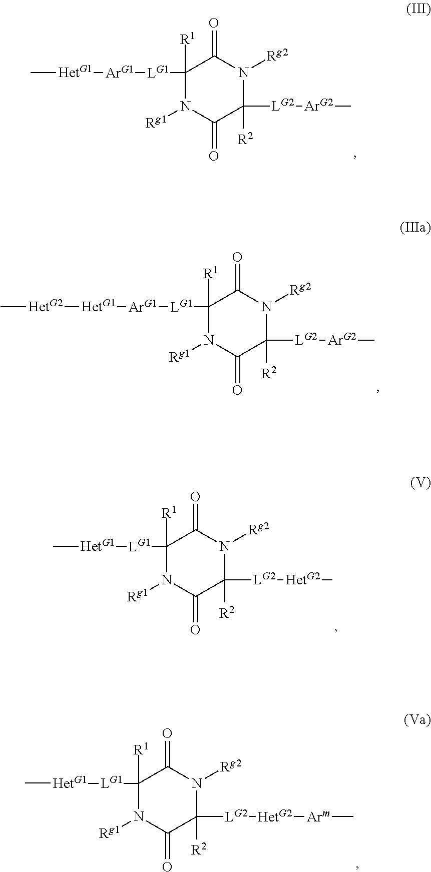

or a salt thereof, in which R^{g1}, R^{g2}, R¹, and R² can be any described herein; and LG L^{G2}, Ar^{G1}, Ar^{G2}, Ar^{m}, Het^{G1}, and Het^{G2} can be any linker described herein. In particular embodiments, each of L^{G1} and L^{G2} is, independently, a covalent bond, an amide bond, or optionally substituted alkylene; each of Ar^{G1} and Ar^{G2} is, independently, optionally substituted arylene or optionally substituted (aryl)(alky)lene; each of Het^{G1}, and Het^{G2} is, independently, optionally substituted heterocyclyldiyl or optionally substituted (heterocyclyl)(alkyl)ene; and Ar^{m} is a polymer segment (e.g., any described herein). In other embodiments, at least one of Ar^{G1}, Ar^{G2}, Het^{G1}, or Het^{G2} includes hydroxyl, carboxyl, amino, amido, an amide bond, an ester bond, oxy, carbonyl, optionally substituted cyclic anhydride, or optionally substituted cyclic imide.

In some embodiments, the subunit includes one or more aryl moieties within the linker. In particular embodiments, the subunit includes a structure of formula (IV), (IVa), (IVb), or (IVc):

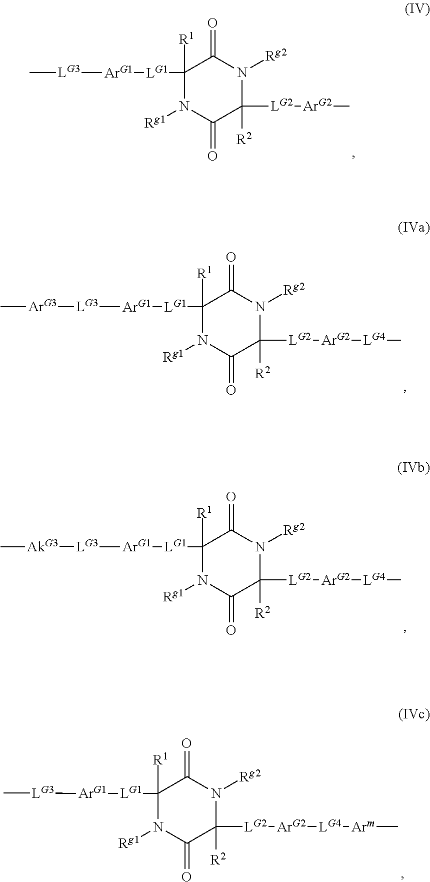

or a salt thereof, in which R^{g1}, R^{g2}, R¹, and R² can be any described herein; and L^{G1}, L^{G2}, L^{G3}, L^{G4}, Ar^{G1}, Ar^{G2}, Ar^{G3}, Ar^{G3}, and Ar^{m} can be any linker described herein. In particular embodiments, each of L^{G1}, L^{G2}, L^{G3}, L^{G4}, and Ar^{G3} is, independently, a covalent bond, an amide bond, or optionally substituted alkylene; each of Ar^{G1}, Ar^{G2}, and Ar^{G3} is, independently, optionally substituted arylene or optionally substituted (aryl)(alky)lene; and Ar^{m} is a polymer segment (e.g., any described herein). In other embodiments, at least one of L^{G3}, L^{G4}, Ar^{G3}, Ar^{G1}, Ar^{G2}, or Ar^{G3} includes hydroxyl, carboxyl, amino, amido, an amide bond, an ester bond, oxy, carbonyl, optionally substituted cyclic anhydride, or optionally substituted cyclic imide.

The subunit can include any useful combination of linkers. In particular embodiments, the subunit includes a structure of formula (VI), (VIa), (VIb), or (VIc):

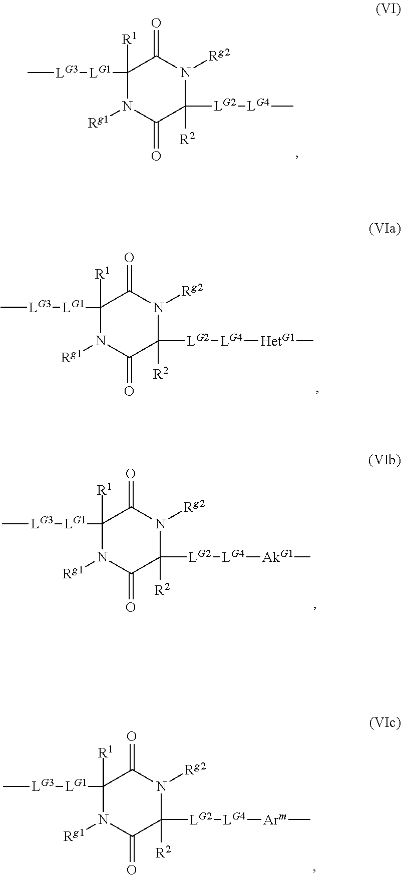

or a salt thereof, in which R^{g1}, R^{g2}, R¹, and R² can be any described herein; and L^{G1}, L^{G2}, L^{G3}, L^{G4}, Ar^{G1}, Ar^{m}, and Het^{G1} can be any linker described herein. In particular embodiments, each of L^{G1}, L^{G2}, L^{G3}, L^{G4}, and Ak^{G1} is, independently, a covalent bond, an amide bond, or optionally substituted alkylene; Ar^{m} is a polymer segment (e.g., any described herein); and Het^{G1} is optionally substituted heterocyclyldiyl or optionally substituted (heterocyclyl)(alkyl)ene. In other embodiments, at least one of L^{G3}, L^{G4}, Ak^{G1}, Het^{G1}, or Ar^{m} includes hydroxyl, carboxyl, amino, amido, an amide bond, an ester bond, oxy, carbonyl, optionally substituted cyclic anhydride, or optionally substituted cyclic imide.

The subunit can include a cyclic proline derivative. In particular embodiments, the subunit includes a structure of formula (VII) or (VIIa):

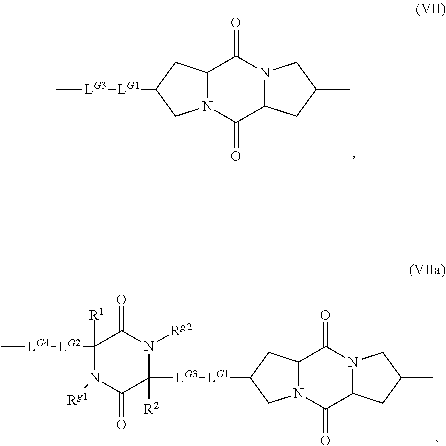

or a salt thereof, in which R^{g1}, R^{g2}, R¹, and R² can be any described herein; and L^{G1}, L^{G2}, L^{G3}, and L^{G4} can be any linker described herein. In other embodiments, at least one of L^{G1}, L^{G3}, or L^{G4} includes hydroxyl, carboxyl, amino, amido, an amide bond, an ester bond, oxy, carbonyl, optionally substituted cyclic anhydride, or optionally substituted cyclic imide.

The subunit can include any useful polymer segment. In particular embodiments, the subunit includes a structure of formula (VIII):

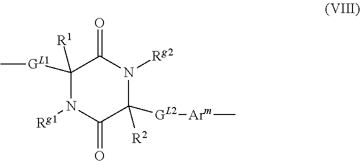

or a salt thereof, in which R^{g1}, R^{g2}, R¹, and R² can be any described herein; G^{L1} and G^{L2} can be any linker described herein; and Ar^{m} can be any polymer segment described herein. In some embodiments, each of G^{L1} and G^{L2} includes, independently, hydroxyl, carboxyl, amino, amido, an amide bond, an ester bond, oxy, carbonyl, optionally substituted cyclic anhydride, optionally substituted cyclic imide, a carbamate bond (e.g., a —O—C(O)—NR^{N1}— bond, in which R^{N1} is H or optionally substituted alkyl), or —NR^{N1}— (in which R^{N1} is H or optionally substituted alkyl).

In any embodiment herein, the linker (e.g., L^{G1}, L^{G2}, L^{G3}, L^{G4}, G^{L}, or G^{L2} in formula (Ia), (Ib), (Id), (Ie), (If), (Ig), (Ih), (Ii), (Ij), (Ik), (IIc), (IId), (III), (IIIa), (IV), (IVa), (IVb), (IVc), (V), (Va), (VI), (VIa), (VIb), (VIc), (VII), (VIIa), or (VIII)) can be selected from any linker herein. Exemplary linkers include a covalent bond, an amide bond, —NR^{N1}— (in which R^{N1} is H or optionally substituted alkyl), a carbamate bond (e.g., a —O—C(O)—NR^{N1}— bond, in which R^{N1} is H or optionally substituted alkyl), an ester bond, oxy, carbonyl, optionally substituted alkylene, optionally substituted alkenylene, optionally substituted heteroalkylene, optionally substituted arylene, optionally substituted (aryl)(alkyl)ene, optionally substituted heterocyclyldiyl, or optionally substituted (heterocyclyl)(alkyl)ene, as well as combinations thereof. In particular non-limiting embodiments, L^{G1}, L^{G2}, L^{G3}, or L^{G4} is a covalent bond, an amide bond, —NR^{N1}—, an ester bond, oxy, carbonyl, optionally substituted alkylene, or optionally substituted heteroalkylene. In other non-limiting embodiments (e.g., in formula (VIII)), each of G^{L1} and G^{L2} is or includes, independently, hydroxyl, carboxyl, amino, amido, an amide bond, an ester bond, oxy, carbonyl, optionally substituted cyclic anhydride, or optionally substituted cyclic imide.

In any embodiment herein, the linker can include an alkyl moiety. In some embodiments (e.g., in formula (IVb) or (VIb)), the linker is any described herein for Ak^{G1} or Ak^{G3}, which can be optionally substituted alkylene or optionally substituted heteroalkylene. Exemplary linkers include -Ak-, -Ak-O—, —O-Ak-, -Ak^{a}-O-Ak^{b}-, -Ak-NR^{N1}—, —NR^{N1}-Ak-, or -Ak^{a}-NR^{N1}-Ak^{b}-, in which each of Ak, Ak^{a}, and Ak^{b} is, independently, optionally substituted alkylene; and R^{N1} is H or optionally substituted alkyl.

In any embodiment herein, the linker can include an aryl moiety. In some embodiments (e.g., in formula (III), (IIIa), (IV), (IVa), (IVb), (IVc), (Va), (VIc), or (VIII)), the linker is any described herein for Ar^{G1}, Ar^{G2}, Ar^{G3}, or Ar^{m}, which can be optionally substituted arylene, optionally substituted (aryl)(alky)lene. Exemplary linkers include —(Ar)ₐ—, —(Ar)ₐ-(Ak)_{c}-, -(Ak)_{c}-(Ar)ₐ—, —(Ar)ₐ—C(O)—(Ar)_{b}—, —(Ar)ₐ—O—(Ar)_{b}—, —(Ar)ₐ—NR^{N1}—(Ar)_{b}—, -(Ak)_{c}-C(O)—(Ar)_{b}—, -(Ak)_{c}-O—(Ar)_{b}—, -(Ak)_{c}-NR^{N1}—(Ar)_{b}—, —(Ar)ₐ—C(O)-(Ak)_{c}-, —(Ar)ₐ—O-(Ak)_{c}-, —(Ar)ₐ—NR^{N1}-(Ak)_{c}-, in which Ar is optionally substituted arylene; Ak is optionally substituted alkylene; R^{N1} is H or optionally substituted alkyl; each a and b is an integer of about 0 to 10 and at least one of a or b is 1 or more; and c is 0 or 1. In particular non-limiting embodiments, Ar is optionally substituted phenylene.

In any embodiment herein, the linker can include a heterocyclyl moiety. In some embodiments (e.g., in formula (Ib), III), (IIIa), (V), (Va), or (VIa)), the linker is any described herein for Het^{G1} or Het^{G2}, which can be optionally substituted heterocyclyldiyl or optionally substituted (heterocyclyl)(alkyl)ene. Exemplary linkers include optionally substituted 2,5-dioxo-1,3-pyrrolidinediyl, optionally substituted 2,5-diketo-3,6-piperazinediyl, optionally substituted 1,3-dioxo-2,3-dihydro-1H-isoindole-2,5-diyl, or optionally substituted 2,8-diketo-1,7-diazatricyclo[7.3.0.03,7]dodecane-4,10-diyl.

In any embodiment herein, the linker can include a polymer segment (e.g., Ar^{m}, as in formula (VIII)). In some embodiments (e.g., in formula (VIII)), the linker is any described herein for Ar^{m}, which can be optionally substituted arylene, optionally substituted (aryl)(alky)lene, an imide subunit, an amic acid subunit, an amide subunit, an arylene subunit, an arylene ether subunit, an arylene ketone subunit, a urethane subunit, a phthalic anhydride subunit, an aliphatic subunit, a cycloalkyl subunit, an ether subunit, or a thioether subunit.

Further exemplary polymer segments include optionally substituted arylene (e.g., —(Ar)ₐ—, —(Ar)ₐ—C(O)—(Ar)_{b}—, —(Ar)ₐ—O—(Ar)_{b}—, or —(Ar)ₐ—NR^{N1}—(Ar)_{b}—); optionally substituted (aryl)(alky)lene (e.g., —(Ar)ₐ-(Ak)_{c}-, -(Ak)_{c}-(Ar)ₐ—, -(Ak)_{c}-C(O)—(Ar)_{b}—, -(Ak)_{c}-O—(Ar)_{b}—, -(Ak)_{c}-NR^{N1}—(Ar)_{b}—, —(Ar)ₐ—C(O)-(Ak)_{c}-, —(Ar)ₐ—O-(Ak)_{c}-, or —(Ar)ₐ—NR^{N1}-(Ak)_{c}-); an imide subunit (e.g., a subunit including an imide bond, such as —[C(O)]_{d}—NR^{Na}—[C(O)]ₑ— or

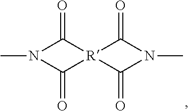

where R′ is an aliphatic or aromatic tetravalent organic moiety that is optionally substituted with a substituent described herein for aryl); an amic acid subunit (e.g., a subunit including (i) a carboxylic acid unit or an ester bond, as defined herein and (ii) a carboxamide unit or an amide bond, as defined herein); an amide subunit (e.g., a subunit including an amide bond, as defined herein); an arylene subunit (e.g., a subunit including an optionally substituted arylene, as defined herein); an arylene ether subunit (e.g., a subunit including an optionally substituted arylene and oxy bonds, such as —(Ar)ₐ—O—(Ar)_{b}—); an arylene ketone subunit (e.g., —(Ar)ₐ—C(O)—(Ar)_{b}—); a urethane subunit (e.g., a subunit including a carbamate bond, such as a —O—C(O)—NR^{N1}— bond); a phthalic anhydride subunit (e.g., a subunit including an anhydride of phthalic acid, such as optionally substituted, multivalent form of 1,3-dioxo-isobenzofuran); an aliphatic subunit (e.g., a subunit including non-aromatic organic moieties, such as optionally substituted alkylene, optionally substituted alkenylene, optionally substituted alkynylene, optionally substituted heteroalkylene, etc.); a cycloalkyl subunit (e.g., a subunit including cycloalkyl moieties, including bivalent, trivalent, or tetravalent forms thereof); an ether subunit (e.g., a subunit including oxy, as defined herein, such as -Ak-O-Ak- or -Ak-O—Ar); a thioether subunit (e.g., a subunit including thio, as defined herein, such as -Ak-S-Ak- or -Ak-S—Ar); a perfluoroalkyl subunit (e.g., —(CF₂)_{f1}—); or a perfluoroalkoxy subunit (e.g., —O(CF₂)_{f1}—, —(CF₂)_{f1}O—, —O(CF₂)_{f1}CF(CF₂)_{f2}—, or >CFO(CF₂)_{f1}CF(CF₂)_{f2}). For any of these linkers, Ar is optionally substituted arylene; Ak is optionally substituted alkylene; R^{N1} is H or optionally substituted alkyl; each a and b is an integer of about 0 to 10 and at least one of a or b is 1 or more; c is 0 or 1; R^{Na} is H, optionally substituted alkyl, optionally substituted aryl, optionally substituted alkaryl, optionally substituted aralkyl, or optionally substituted heterocyclyl; and each f1 and f2 is, independently, an integer of 1 to about 16. In addition, an aliphatic or aromatic tetravalent organic moiety can be, e.g., optionally substituted alkane-tetrayl, optionally substituted benzene-1,2,4,5-tetrayl, optionally substituted naphthalene-2,3,6,7-tetrayl, optionally substituted anthracene-2,3,6,7-tetrayl, optionally substituted phenanthrene-1,8,9,10-tetrayl, >Ar*—Ar*<, >Ar*—O—Ar*<, >Ar*—C(O)—Ar*<, >Ar*—S—Ar*<, >Ar*—S(O)₂—Ar*<, >Ar*—Si(CH₃)₂-Ar*<, >Ar*—O—Ar—O—Ar—O—Ar*<, >Ar*—O—Ar—C(O)—Ar—O—Ar*<, >Ar*—O—Ar—S—Ar—O—Ar*<, >Ar*—O—Ar—S(O)₂—Ar—O—Ar*<, or >Ar*—O—Ar—Si(CH₃)₂—Ar—O—Ar*<, in which Ar* is an optionally substituted trivalent arylene, such as benzene-1,3,4-triyl, and in which Ar is optionally substituted divalent arylene).

Reactive moieties can also be characterized as a polymerizable group. A polymerizable group includes groups that form homopolymers or copolymers. In a first embodiment, the polymerizable group can form predominately homopolymers, meaning that the compound A forms polymers symbolized as -(A-A-A)X—, wherein x is an integer. These groups are defined as homopolymerizable. Examples of such groups are unsaturated groups, such as vinyl and allyl groups, oxiranes (ethylene oxides or epoxides), aziridines (ethylene imines), oxetanes. In another embodiment, the polymerizable group is copolymerizable, i.e., a second compound B is required to form polymers -(A-B-A-B)X—, wherein x is an integer. Examples of such groups are carboxylic acids, hydroxyl groups, amino groups, and thiol groups; and examples for the respective copolymer monomer would be diols or diamines, diacids, diacid anhydrides, isocyanates, and di-isocyanates.

In one embodiment, the polymerizable group can be selected from a vinyl group, an allyl group, hydroxyl, carboxyl, amino, cyclic anhydride, cyclic imide, or a combination thereof.

In another embodiment, at least 35 wt. %, such as at least 40 wt. %, at least 45 wt. %, at least 50 wt. %, at least 55 wt. %, at least 60 wt. %, at least 65 wt. %, at least 70 wt. %, at least 75 wt. %, at least 80 wt. %, or at least 85 wt. % of the compound or the composition is comprised by the moiety. In another embodiment, not more than 98 wt. %, such as not more than 96 wt. %, not more than 95 wt. %, not more than 94 wt. %, not more than 92 wt. %, or not more than 90 wt. % of the compound or the composition are comprised by the moiety. In yet one further embodiment, the moiety of the compound or the composition has weight percentage in the range between 30 wt. % to 99.5 wt. %, such as 40 wt. % to 98 wt. %, or even 50.5 wt. % to 96 wt. %.

In yet one further embodiment, at least 60 wt. %, at least 65 wt. %, at least 70 wt. %, at least 75 wt. %, at least 80 wt. %, at least 85 wt. %, or at least 88 wt. % of the compound or the composition are comprised by the sum of weight percentages of the moiety and the polymerizable group. In another embodiment, not more than 99.9 wt. %, such as not more than 99 wt. %, not more than 98 wt. %, not more than 96 wt. %, not more than 94 wt. %, not more than 92 wt. %, not more than 90 wt. %, not more than 85 wt. %, or not more than 80 wt. % of the compound or the composition are comprised by the sum of weight percentages of the moiety and the polymerizable group. In yet one further embodiment, the sum of weight percentages of the moiety and the polymerizable group can range between 55 wt. % to 99.99 wt. %, such as 65 wt. % to 99 wt. %, or 75 wt. % to 98 wt. %.

In any of the formulas herein, R^{g1} and R^{g2} can be H, optionally substituted alkyl, haloalkyl, alkoxyalkyl, or any combination thereof. Other non-limiting R^{g1} and R^{g2} groups include, independently for each occasion, hydrogen or C₁₋₂₀ straight or branched alkyl chains, such as methyl, ethyl, n-propyl, 2-propyl, 1-butyl, 2-butyl, 2-methylpropyl, pentyl, 2-methylbutyl, 2,2-dimethylpropyl, hexyl, 2-methylpentyl, 3-methylpentyl, 2,2-dimethylbutyl, 2,3-dimethylbutyl, heptyl, 2-methylhexyl, 3-methylhexyl, 2,2-dimethylpentyl, 2,3-dimethylpentyl, 2,4-dimethylpentyl, 3,3-dimethylpentyl, 3-ethylpentyl, 2,2,3-trimethylbutyl, octyl, 2-methylheptyl, 3-methylheptyl, 4-methylheptyl, 5-methylheptyl, 6-methylheptyl, 2-ethylhexyl, 3-ethylhexyl, 4-ethylhexyl, 2,3-dimethylhexyl, 2,4-dimethylhexyl, 2,5-dimethylhexyl, 3,4-dimethylhexyl, 3,5-dimethylhexyl, 4,5-dimethylhexyl, 2-propylpentyl, nonyl, decyl, undecyl, dodecyl, tridecyl, tetradecyl, pentadecyl, hexadecyl, heptadecyl, octadecyl, nonadecyl, and icosyl.

In a further embodiment, the foregoing compound or composition has a bio-based carbon content of at least 10%, such as at least 15%, at least 20%, at least 25%, at least 30%, or at least 35% as determined by ASTM D6866. Bio-based carbon content as defined herein is the percentage of carbons from renewable or biogenic sources, such as plants or animals over the total number of carbons in the compound.

For example, the following cyclic derivative is prepared from bio-sourced tyrosine and petrochemically epichlorohydrin:

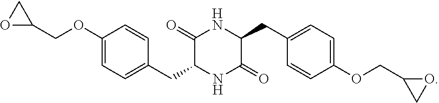 [SMILES: O=C1N[C@H](Cc2ccc(OCC3CO3)cc2)C(=O)N[C@H]1Cc1ccc(OCC2CO2)cc1]

Then, 16 carbon atoms are bio-based and 6 carbon atoms are petrochemically sourced. Upon analysis according to ASTM D6866, this compound has a bio-based carbon content of 16/(16+6)=72.7%.

Additional cyclic derivatives and functionalized steroids are provided below in Table 1.

TABLE 1
Non-limiting cyclic derivatives and functionalized steroids

| Compound No. | Structure |
|---|---|
| I-1 | 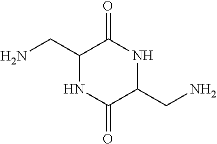 [SMILES: NCC1NC(=O)C(CN)NC1=O] |
| I-2 | 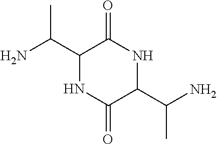 |
| I-3 | 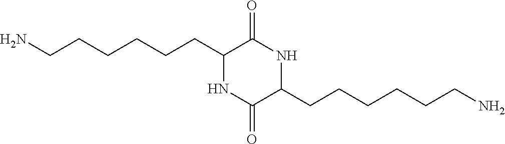 [SMILES: NCCCCCCC1NC(=O)C(CCCCCCN)NC1=O] |
| I-4 | 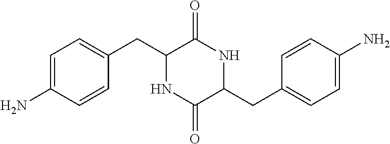 [SMILES: Nc1ccc(CC2NC(=O)C(Cc3ccc(N)cc3)NC2=O)cc1] |
| I-5 | 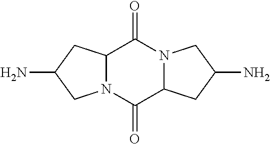 [SMILES: NC1CC2C(=O)N3CC(N)CC3C(=O)N2C1] |
| I-6 | 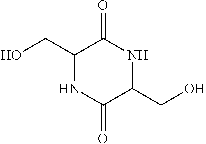 [SMILES: O=C1NC(CO)C(=O)NC1CO] |
| I-7 | 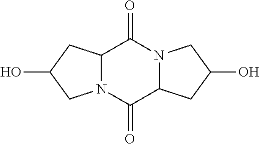 [SMILES: O=C1C2CC(O)CN2C(=O)C2CC(O)CN12] |
| I-8 | 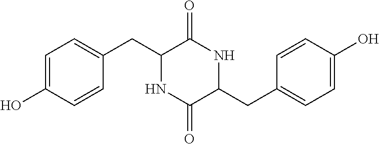 [SMILES: O=C1NC(Cc2ccc(O)cc2)C(=O)NC1Cc1ccc(O)cc1] |
| I-9 | 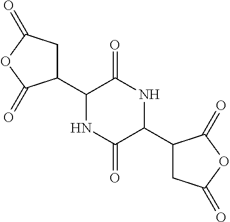 [SMILES: O=C1CC(C2NC(=O)C(C3CC(=O)OC3=O)NC2=O)C(=O)O1] |
| I-10 | 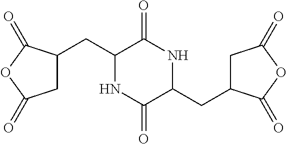 [SMILES: O=C1CC(CC2NC(=O)C(CC3CC(=O)OC3=O)NC2=O)C(=O)O1] |
| I-11 | 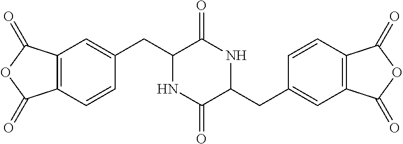 [SMILES: O=C1OC(=O)c2cc(CC3NC(=O)C(Cc4ccc5c(c4)C(=O)OC5=O)NC3=O)ccc21] |
| II-1 | 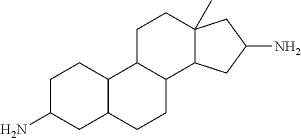 [SMILES: CC12CCC3C4CCC(N)CC4CCC3C1CC(N)C2] |
| II-2 | 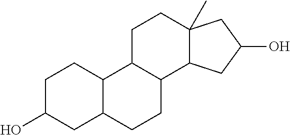 [SMILES: CC12CCC3C4CCC(O)CC4CCC3C1CC(O)C2] |
| II-3 | 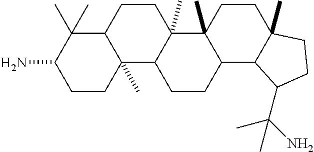 [SMILES: CC(C)(N)C1CC[C@@]2(C)CC[C@]3(C)C(CCC4[C@@]5(C)CC[C@H](N)C(C)(C)C5CC[C@]43C)C12] |
| II-4 | 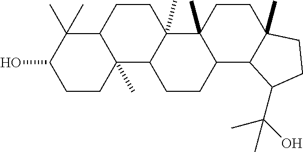 [SMILES: CC(C)(O)C1CC[C@@]2(C)CC[C@]3(C)C(CCC4[C@@]5(C)CC[C@H](O)C(C)(C)C5CC[C@]43C)C12] |

The cyclic derivatives herein can be prepared in any useful manner, such as by providing a first biomolecule and a second biomolecule and forming a dimer between the first and second biomolecules. The first and second biomolecules can be any herein, including, e.g., amino acids, such as glycine, vinylglycine, 2-allylglycine, alanine, valine, leucine, isoleucine, methionine, phenylalanine, 4-aminophenylalanine, tyrosine, tryptophan, serine, threonine, asparagine, glutamine, cystine, homocystine, cysteine, homocysteine, selenocysteine, proline, hydroxyproline, arginine, histidine, lysine, aspartic acid, glutaminic acid, 4-(2-amino-2-carboxyethyl)-1,2-benzenedicarboxylic acid, 1-aminopropane-1,2,3-tricarboxylic acid, 4-amino-1,2,4-butanetricarboxylic acid, and well as any of these including an optionally substituted alkenyl, amino, and/or hydroxyl. Additional non-limiting biomolecules are further described herein.

The dimer can be further functionalized (e.g., to include one or more linkers and/or reactive moieties). The methods herein can further include hydroaminating an alkenyl group in the presence of a nitrogen-containing reactant (e.g., an amine, such as NR^{N1}R^{N2}R^{N3}, in which each of R^{N1}, R^{N2}, and R^{N3} is, independently, H, optionally substituted alkyl, optionally substituted alkenyl, optionally substituted alkynyl, optionally substituted aryl, or a leaving group, such as an ester group).

In yet other embodiments, the cyclic derivatives herein can be prepared by processes analogous to those established in the art, for example, by the reaction sequences shown in Schemes 1-2.

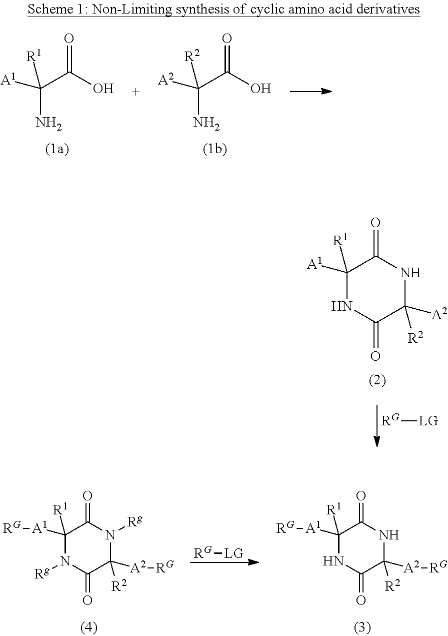

As seen in Scheme 1, amino acids (1a, 1b) can be provided, in which R¹ and R² can be H, alkyl, any described herein for R¹ and R²; and in which A¹ and A² can be an amino acid side chain or a functionalized form thereof. Substituents within the amino acid can be optionally protected with a protecting group (e.g., an N-protecting group for amino or an O-protecting group for hydroxyl) or can be optionally functionalized to provide a better leaving group (e.g., an alkylating agent for oxygen to provide an alkoxy leaving group). Amino acids (1a, 1b) can be the same or different. Furthermore, such amino acids can be optionally provided by a biological resource.

Cyclic amino acids (2) can be provided by dimerization and cyclization of the amino acids (1a, 1b) in the presence of a solvent (e.g., ethylene glycol). If desired, dimers can first be formed to promote internal cyclization within the dimer. Dimerization can be performed in any useful manner (e.g., with use of protecting groups); and subsequent cyclization can optionally be performed under catalytic conditions (e.g., with subsequent deprotection chemistry to remove protecting groups).

Reactive moieties can be provided within the side chains of A¹ and A² of amino acids (1a, 1b, respectively), prior to dimerization. Alternatively, cyclic amino acid (2) can be functionalized with R^{G}-LG to provide a cyclic derivative (3), in which R^{G} is or includes a reactive moiety (e.g., any described herein, such as for R^{G}, R^{G1}, or R^{G2}) and LG is a leaving group (e.g., halo).

Further functionalization of compound (3) can provide another cyclic derivative (4), in which nitrogen atoms of the diketopiperazine can include be further substituted. Here, compound (3) can be functionalized with R⁸-LG to provide cyclic derivative (4), in which R^{g} can be any described herein (e.g., such as for R^{g1} and R^{g2}).

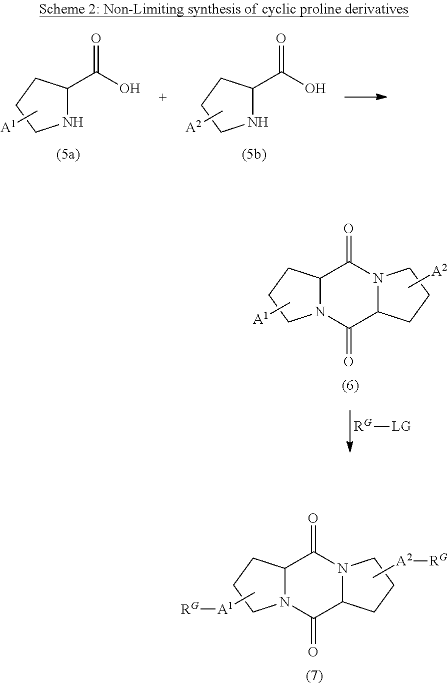

As seen in Scheme 2, proline derivatives (5a, 5b) can be provided, in which A and A² can be an amino acid side chain or a functionalized form thereof. In one instance, A and A² includes hydroxyl for a hydroxyproline derivative (e.g., 4-hydroxyproline). Amino acids (5a, 5b) can be the same or different and can be optionally provided by a biological resource.

Cyclic amino acids (6) can be provided by dimerization and cyclization of the amino acids (5a, 5b) in the presence of a solvent (e.g., ethylene glycol). Reactive moieties can be provided within the side chains of A¹ and A² of proline derivatives (5a, 5b, respectively), prior to dimerization. Alternatively, reactive moieties can be provided by functionalizing the cyclic amino acid (6) with R^{G}-LG to provide a cyclic derivative (7), in which R^{G} is or includes a reactive moiety (e.g., any described herein, such as for R^{G}, R^{G1}, or R^{G2}) and LG is a leaving group (e.g., halo).

The cyclic derivatives herein can be prepared in any useful manner, such as by providing a first biomolecule and a second biomolecule and forming a dimer between the first and second biomolecules. The first and second biomolecules can be any herein, including, e.g., amino acids, such as glycine, vinylglycine, 2-allylglycine, alanine, valine, leucine, isoleucine, methionine, phenylalanine, 4-aminophenylalanine, tyrosine, tryptophan, serine, threonine, asparagine, glutamine, cystine, homocystine, cysteine, homocysteine, selenocysteine, proline, hydroxyproline, arginine, histidine, lysine, aspartic acid, glutaminic acid, 4-(2-amino-2-carboxyethyl)-1,2-benzenedicarboxylic acid, 1-aminopropane-1,2,3-tricarboxylic acid, 4-amino-1,2,4-butanetricarboxylic acid, and well as any of these including an optionally substituted alkenyl, amino, and/or hydroxyl. Additional non-limiting biomolecules are further described herein.

As described herein, cyclic derivatives can be employed as a subunit within a polymer or copolymer. Non-limiting structures of subunits are provided below in Table 2 (e.g., in which n is any integer, such as from 1 to 100). Such subunits can provide any useful polymer or copolymer, such as a polyamic acid, a polyimide, a polyamide, a polyester, a polyurethane, or combinations thereof.

TABLE 2
Non-limiting subunits

| Compound No. | Structure |
|---|---|
| VIII-1 | 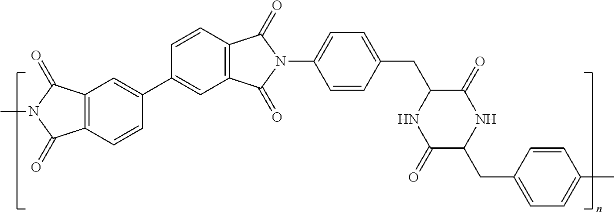 |
| VIII-2 | 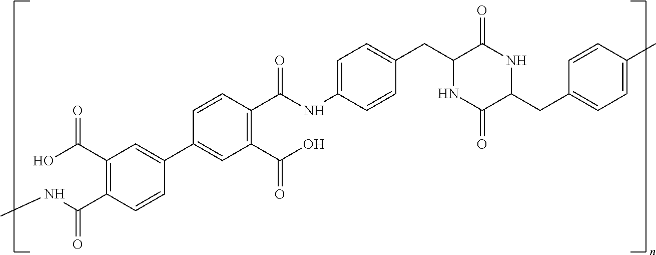 |
| VIII-3 | 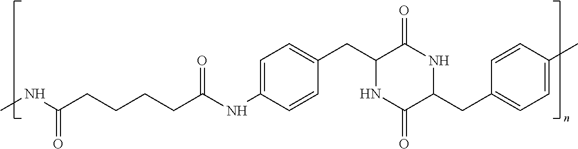 |
| VIII-4 | 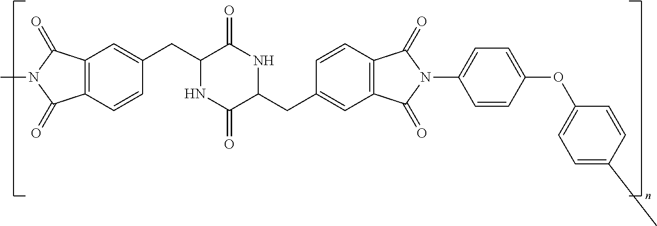 |
| VIII-5 | 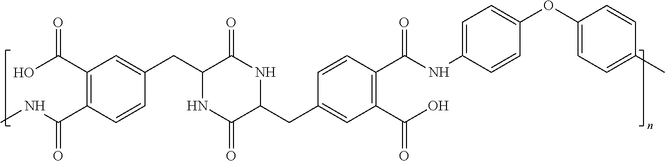 |
| VIII-6 | 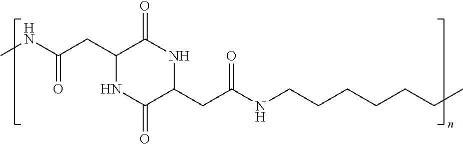 |
| VIII-7 | 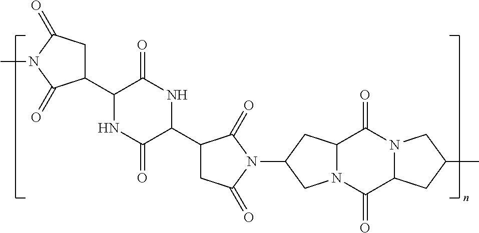 |
| VIII-8 | 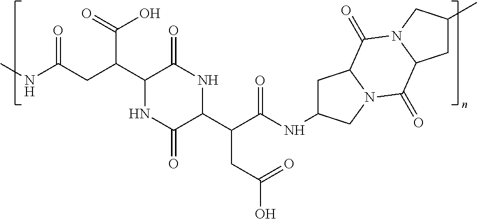 |
| VIII-9 | 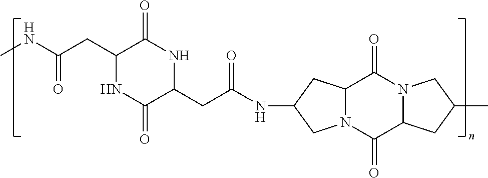 |

Methods of preparing polymers having a subunit (e.g., any described herein) can include reacting a cyclic derivative (e.g., any described herein) with any useful monomer (e.g., a petroleum-based monomer or another cyclic derivative, such as any herein). Non-limiting monomers include a diamine (e.g., a petroleum-based diamine) with any cyclic derivative herein (e.g., a cyclic derivative including an optionally substituted cyclic anhydride, optionally substituted cyclic imide, or other reactive group); a diacid (e.g., a petroleum-based diacid) or a tetraacid (e.g., a petroleum-based diacid or tetraacid) with any cyclic derivative herein (e.g., a cyclic derivative including an optionally substituted cyclic anhydride, optionally substituted cyclic imide, or other reactive group); a dianhydride (e.g., a petroleum-based dianhydride) with any cyclic derivative herein (e.g., a cyclic derivative with amino, hydroxyl, or other reactive group); or a first cyclic derivative (e.g., any herein) including an optionally substituted cyclic anhydride or an optionally substituted cyclic imide, which is reacted with a second cyclic derivative (e.g., any herein) including hydroxyl, carboxyl, or amino.

Non-limiting diamines include, e.g., NR^{N1}R^{N2}-Ak-NR^{N3}R^{N4}, NR^{N1}R^{N2}-Cy-NR^{N3}R^{N4}, NR^{N1}R^{N2}—Ar—NR^{N3}R^{N4}, or NR^{N1}R^{N2}—[Ar—X]ᵣ—Ar—NR^{N3}R^{N4}, in which each Ak is, independently, optionally substituted alkylene; Cy is a multivalent (e.g., bivalent) form of a cycloalkyl group, as described herein; each Ar is, independently, optionally substituted arylene; each X is, independently, a covalent bond, oxy, thio, alkylene, or carbonyl; each of R^{N1}, R^{N2}, R^{N3}, and R^{N4} is, independently, H, optionally substituted alkyl, or an N-protecting group; and r is an integer from 0 to 4.

Examples of diamines include 4,4′-oxydianiline (4,4′-ODA); 3,4′-oxydianiline (3,4′-ODA); 4,4′-(4,4′-isopropylidenediphenyl-1,1′-diyldioxy)dianiline; m-phenylenediamine (MPD); p-phenylenediamine (PPD); 2,2-bis[4-(4-aminophenoxy)phenyl] propane (BAPP); 4,4′-methylene dianiline (MDA); 4,4′-(4-aminophenoxy)biphenyl (4BPDA); 2,2-bis[4-(4-aminophenoxy)phenyl]-hexafluoropropane (BDAF); 4,4′-[1,3-phenylenebis(1-methyl-ethylidene)] bisaniline (Bisaniline-M); 4,4′-[1,4-phenylenebis(1-methyl-ethylidene)] bisaniline (Bisaniline-P); 1,2-diaminoethane (1,2-DAE); 1,3-diaminopropane (1,3-DAP); 1,4-diaminobutane (1,4-DAB); 1,5-diaminopentane (1,5-DAP); 1,6-diaminohexane (1,6-HMDA), 1,7-diaminoheptane; 1,8-diaminooctane; 1,9-diaminononane; 1,10-diaminodecane; 1,11-diaminoundecane; 1,12-diaminododecane; N-(3-aminopropyl)-1,4-butadiamine; N,N′-bis(3-aminopropyl)-1,4-butanediamine; N-(3-aminopropyl)-1,3-propanediamine; N1-(3-(3-aminopropylamino)propyl)butane-1,4-diamine; 1,4-diamino-2-methylcyclohexane; 1,4-diamino-2-ethylcyclohexane; 1,4-diamino-2-n-propylcyclohexane; 1,4-diamino-2-isobutylcyclohexane; and 1,4-diamino-2-tert-butylcyclohexane. Yet other diamines include, e.g., a cyclic derivative having a structure of formula (I), in which both G¹ and G² is or includes amino, optionally substituted aminoalkyl, or optionally substituted aminoaryl.

Non-limiting diacids and tetraacids include HOC(O)-Ak-C(O)OH or

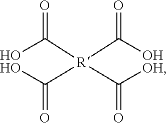

in which Ak is optionally substituted alkylene and R′ is an aliphatic or aromatic tetravalent organic moiety that is optionally substituted with a substituent described herein for aryl. Examples of aliphatic or aromatic tetravalent groups include optionally substituted alkane-tetrayl (a tetravalent form of alkyl), optionally substituted benzene-1,2,4,5-tetrayl, optionally substituted naphthalene-2,3,6,7-tetrayl, optionally substituted anthracene-2,3,6,7-tetrayl, optionally substituted phenanthrene-1,8,9,10-tetrayl, >Ar*—Ar*<, >Ar*—O—Ar*<, >Ar*—C(O)—Ar*<, >Ar*—S—Ar*<, >Ar*—S(O)₂—Ar*<, >Ar*—Si(CH₃)₂—Ar*<, >Ar*—O—Ar—O—Ar—O—Ar*<, >Ar*—O—Ar—C(O)—Ar—O—Ar*<, >Ar*—O—Ar—S—Ar—O—Ar*<, >Ar*—O—Ar—S(O)₂—Ar—O—Ar*<, or >Ar*—O—Ar—Si(CH₃)₂—Ar—O—Ar*<, in which Ar* is an optionally substituted trivalent arylene, such as benzene-1,3,4-triyl, and in which Ar is optionally substituted divalent arylene.

Examples of diacids and tetraacids include 1,4-butanedioic acid (succinic acid), 1,6-hexanedioic acid (adipic acid), 1,7-heptanedioic acid (pimelic acid), 1,8-octanedioic acid (suberic acid), 1,9-nonanedioic acid (azelaic acid), 1,10-decanedioic acid (sebacic acid), 1,11-undecanedioic acid, 1,12-dodecanedioic acid (1,10-decanedicarboxylic acid), 1,13-tridecanedioic acid (brassylic acid) 1,14-tetradecanedioic acid (1,12-dodecanedicarboxylic acid), 1,4-cyclohexanedicarboxylic acid (CHDA), 1,3-cyclohexanedicarboxylic acid (CHDA), and phthalic acid. Yet other diacids include, e.g., a cyclic derivative having a structure of formula (I), in which both G¹ and G² is or includes carboxyl, optionally substituted carboxyalkyl, or optionally substituted carboxyaryl.

Non-limiting dianhydrides include

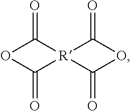

where R′ is an aliphatic or aromatic tetravalent organic moiety, as provided herein for a tetraacid. Examples of dianhydrides include, e.g., 3,3′,4,4′-biphenyltetracarboxylic dianhydride (s-BPDA); 2,2′,4,4′-biphenyltetracarboxylic acid dianhydride (BPDA); 2,3,3′,4′-biphenyltetracarboxylic acid dianhydride (a-BPDA); 4,4′-oxydiphthalic anhydride (ODPA); pyromellitic dianhydride (PMDA); benzophenone-3,3′,4,4′-tetracarboxylic dianhydride (BTDA); isophthaloyl bisphthalic dianhydride (IPDA); 4,4′-(hexafluoroisopropylidene) diphthalic anhydride (6FDA); naphthalenetetracarboxylic dianhydride (NTDA); triptycene tetracarboxylic dianhydride (TDA and TPDA); 3,3′,4,4′-diphenylsulfone tetracarboxylic dianhydride (DSDA); 4,4′-bisphenol A dianhydride or 4,4′-(4,4′-isopropylidenediphenoxy) bis(phthalic anhydride) (BPADA); hydroquinone diphthalic anhydride (HQDEA); 2,3,4,5-tetrahydrofurantetracarboxylic dianhydride; 5-(2,5-dioxotetrahydrofural)-3-methyl-3-cyclohexane-1,2-dicarboxylic dianhydride; 1,2,4,5-cyclohexanetetracarboxylic acid dianhydride (H-PMDA); 3,4-dicarboxy-1,2,3,4-tetrahydro-1-naphthalenesuccinic acid; 3,3′,4,4′-bicyclohexyltetracarboxylic acid dianhydride (H-BPDA); 3,3′,4,4′-diphenylsulphone tetracarboxylic dianhydride; 3,3′,4,4′-diphenylpropane 2,2-tetracarboxylic dianhydride; 2,2-bis(3,4-dicarboxyphenyl)propane dianhydride; 1,4-bis(3,4-dicarboxybenzoyl)benzene dianhydride; 1,3-bis(3,4-dicarboxybenzoyl)benzene dianhydride; bis(3,4-dicarboxyphenyl) thioether dianhydride; spiro bisindane diether anhydride; bis-phenol A bisether-4-phthalic dianhydride; 1,4,5,8-naphthalenetetracraboxylic dianhydride; 2,3,6,7-naphthalenetetracarboxylic dianhydride; 3,3′,4,4′-dimethyldiphenylsilanetetracarboxylic dianhydride; 3,3′,4,4′-tetraphenyl silanetetracarboxylic dianhydride; p-phenylene-bis(triphenylphthalic acid)dianhydride; and m-phenylene-bis(triphenylphthalic acid)dianhydride. Yet other dianhydrides include, e.g., a cyclic derivative having a structure of formula (I), in which both G¹ and G² is or includes optionally substituted cyclic anhydride.

### Biomolecules, Including Amino Acids, Steroids, and Derivatives Thereof

As described herein, the compositions and methods herein can employ biomolecules, which can be further undergo dimerization, cyclization, and/or functionalization. Non-limiting biomolecules include amino acids, such as glycine, vinylglycine, allylglycine, alkenylglycine, tyrosine, O-allyltyrosine, O-alkenyltryrosine, tryptophan, allyltryptophan, alkenyltryptophan, phenylalanine, allylphenylalanine, alkenylphenylalanine, proline, hydroxyproline (e.g., 4-hydroxyproline), serine, alanine, valine, leucine, isoleucine, methionine, phenylalanine, 4-aminophenylalanine, threonine, asparagine, glutamine, cystine, homocystine, cysteine, homocysteine, selenocysteine, arginine, histidine, lysine, aspartic acid, glutaminic acid, 4-(2-amino-2-carboxyethyl)-1,2-benzenedicarboxylic acid, 1-aminopropane-1,2,3-tricarboxylic acid, and 4-amino-1,2,4-butanetricarboxylic acid. Other non-limiting biomolecules include steroids, such as betulin, pregnane, and others.

Yet other non-limiting biomolecules can include, e.g., derivatives of any amino acids including an alkenyl, amino, carboxyl, or hydroxyl moiety. In particular embodiments, the biomolecule is an L-amino acid or a functionalized L-amino acid having an alkenyl, amino, carboxyl, or hydroxyl moiety.

Biomolecules can be formed in any useful manner. In one instance, the biomolecules are produced from yeast, gram positive bacteria, gram negative bacteria, or fungi. In other embodiments, amino acids, as well as derivatives thereof and/or dimers thereof, and steroids are produced biologically by way of fermentation. In yet other embodiments, amino acid dimers are produced by chemical means using petro-based starting materials.

### Applications

The compositions herein can be employed as ingredients and/or monomers in any useful application. Exemplary, non-limiting applications include adhesives, coatings, films, and plastics. Such applications can include materials for use in constructing electronics, industrial adhesives, architectural adhesives and coatings, civil engineering adhesives and coatings, transportation adhesives and coatings, handheld devices, electronic devices, energy storage devices, energy generation devices, personal electronics (e.g., smart phones, laptops, or tablets), displays, sensors, semi-conductor materials (e.g., such as in chip patterning, manufacturing, and packaging), packages, and the like.

In some embodiments, the compositions described herein can be used in electronics applications, such as, but not limited to microelectronic components or electronic displays. For example, the composition can be used as a transparent base material in the display. In various embodiments, the compositions can be used in waveguides, organic light emitting diodes, electronic paper, liquid crystal displays, electroluminescent display, thin film transistors, flexible electronics, wearable electronics, and as a dielectric material. In certain embodiments, the compositions described herein can be used in solar cells, e.g., where the composition is a transparent substrate in the solar cell.

In particular embodiments, the compositions are used as, or incorporated into, a film that has a thickness between 10 nm and 1 cm (inclusive). In various embodiments, the film thickness is on the order of 10, 50, 100, 200, 300, 400, 500 600, 700, 800, or 900 nm, or 1, 10, 50, 100, 200, 300, 400, 500, 600, 700, 800, or 900 mm, or 1 cm. In some embodiments, the film thickness falls within a range bounded by any of these values, e.g., 50 nm to 900 mm, 200 nm to 700 mm, 500 nm to 500 mm (including the endpoints).

Yet other applications include use of the composition as a polymer curative, a resin, a monomer for a polymer or a copolymer, and the like. The composition can be provided in any useful form, such as a film, a composite structure, a bulk structure, a fiber, or a particle. The composition can optionally include one or more hardeners for use with the cyclic derivatives. Non-limiting hardeners include, e.g., diamines (such as 1,4-diamino butane (DAB), 1,13-diamino-4,7,10-trioxatridecane (TDD), or any herein). If desired, the composition can also include an accelerator, such as tris(dimethylaminomethyl)phenol, or other additives (e.g., resorcinol diglycidyl ether).

In particular embodiments, the compositions herein can undergo bio-triggered degradation for debonding of adhesives, coatings, and composites. Degradation can be triggered, e.g., by employing one or more proteases, hydrolases, peptidases, and the like. Such degradation can be promoted by providing a microbe or a supernatant derived from such microbes (e.g., such as Streptomyces flavovirens, Paenibacillus chibensis, Leifsonia sp., Bacillus sp., Rhizobium sp., Paenibacillus sp., and Microbacterium sp.). In use, the final enzymatic breakdown product can be the starting amino acid, such that the cyclic derivatives, as well as any composition including such derivatives, can exhibit biodegradability. In one non-limiting instance, a cyclic derivative (e.g., any herein) can be employed as a film, a coating, or an adhesive, which can be de-bonded on demand with an engineered microbe and be reverted back to benign starting materials (amino acids).

In some embodiments, the present disclosure encompasses methods for manufacturing any use herein (e.g., an adhesive, a coating, a film, a plastic, a composite, an electronic device, an energy storage device, an energy generation device, and the like) by applying a composition herein (e.g., any foregoing compound) in the assembly of the adhesive, the coating, the film, the plastic, the composite, the electronic device, the energy storage device, or the energy generation device. In other embodiments, the composition is provided as a polymer curative.

The composition can possess any useful property. In one embodiment, the optical transmittance of the composition is at least 60% at 290 nm, 300 nm, 350 nm, 400 nm, 450 nm, 550 nm, and above. In various embodiments, the composition has an optical transmittance of at least 60%, 65%, 70%, 75%, 80%, 85%, 90%, 95%, 98%, or 99% at 550 nm and above. In various embodiments, such compositions have a transmittance of at least 60%, 65%, 70%, 75%, 80%, 85%, 90%, 95%, 98%, or 99% at 500 nm and above. In various embodiments, such compositions have a transmittance of at least 60%, 65%, 70%, 75%, 80%, 85%, 90%, 95%, 98%, or 99% at 450 nm and above. In various embodiments, such compositions have a transmittance of at least 60%, 65%, 70%, 75%, 80%, 85%, 90%, 95%, 98%, or 99% at 400 nm and above. In various embodiments, such compositions have a transmittance of at least 60%, 65%, 70%, 75%, 80%, 85%, 90%, 95%, 98%, or 99% at 350 nm and above. In various embodiments, such compositions have a transmittance of at least 60%, 65%, 70%, 75%, 80%, 85%, 90%, 95%, 98%, or 99% at 300 nm and above. In various embodiments, such compositions have a transmittance of at least 60%, 65%, 70%, 75%, 80%, 85%, 90%, 95%, 98%, or 99% at 270 nm and above.

In various embodiments, the compositions described herein have a birefringence in the range of −0.005 and +0.005, −0.002 and +0.002, −0.001 and +0.001, or −0.0005 and +0.0005 (inclusive of these endpoints).

In particular embodiments, the composition is optically clear and perceived visually to be devoid of color. In some embodiments, the composition has a UV cutoff less than about 450 nm, 400 nm, 350 nm, 300 nm, 270 nm, or lower. In other embodiments, the composition has a yellowness index less than about 2, 1.8, 1.6, 1.5. 1.4, or 1. In yet other embodiments, the composition has a modulus less than about 12 GPa, 10 GPa, or less. In some embodiments, the composition has an elongation less than about 350%, 300%, 250%, 200%, 150%, or less.

In other embodiments, the composition is soluble in an organic solvent (e.g., an organic polar solvent, such as N-methyl-2-pyrrolidone (NMP), N,N-dimethylacetamide (DMAc), cyclopentanone, chloroform, and combinations thereof). In particular, the composition can be dissolved in the solvent to produce a solution that is processed into a material (e.g., a film, a fiber, a coating, an adhesive, a composite, a bulk structure, or a particle). Processing can include solution cast lines, ink jetting, dip coating, spraying, spin coating, casting, blow molding, extrusion, pultrusion, injection molding, melt-processing, and/or electrospinning.

Many different aspects and embodiments are possible. Some of those aspects and embodiments are described herein. After reading this specification, skilled artisans will appreciate that those aspects and embodiments are only illustrative and do not limit the scope of the present invention. Embodiments may be in accordance with any one or more of the items as listed below.

Embodiment 1. A composition including a structure having formula (I):

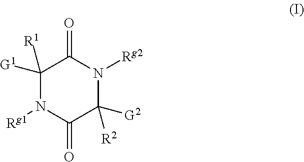

or a salt thereof, wherein: each of G¹ and G² includes, independently, hydroxyl, carboxyl, amino, amido, optionally substituted cyclic anhydride, or optionally substituted cyclic imide; each of R¹ and R² is, independently, H or optionally substituted alkyl; each of R^{g1} and R^{g2} is, independently, optionally substituted alkyl, optionally substituted aryl, optionally substituted alkaryl, or optionally substituted aralkyl; R^{g1} and G¹, taken together with the nitrogen to which R^{g1} is bound, can optionally form an optionally substituted heterocyclyl; and R^{g2} and G², taken together with the nitrogen to which R^{g2} is bound, can optionally form an optionally substituted heterocyclyl.

Embodiment 2. The composition of embodiment 1, wherein: G¹ is -L^{G1}-R^{G1} and G² is -L^{G2}-R^{G2}; each of L^{G1} and L^{G2} is, independently, a covalent bond, an amide bond, optionally substituted alkylene, optionally substituted alkenylene, optionally substituted heteroalkylene, optionally substituted heterocyclyldiyl, or optionally substituted (heterocyclyl)(alkyl)ene; and each of R^{G1} and R^{G2} is, independently, hydroxyl, optionally substituted hydroxyalkyl, optionally substituted hydroxyaryl, carboxyl, amino, optionally substituted aminoalkyl, optionally substituted aminoaryl, amido, optionally substituted amidoalkyl, optionally substituted cyclic anhydride, or optionally substituted cyclic imide.

Embodiment 3. The composition of embodiment 2, wherein the optionally substituted cyclic anhydride has a structure of:

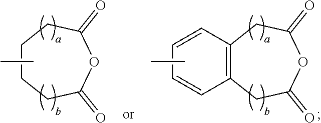

and wherein each of a and b, independently, is an integer of from 0 to 3.

Embodiment 4. The composition of embodiment 2, wherein the optionally substituted cyclic imide has a structure of:

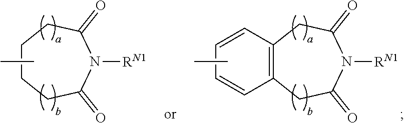

wherein each of a and b, independently, is an integer of from 0 to 3; and wherein R^{N1} is H, optionally substituted alkyl, optionally substituted aryl, or optionally substituted aralkyl.

Embodiment 5. The composition of embodiment 1, wherein the composition includes a structure having formula (Ia):

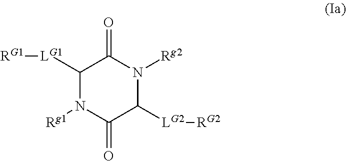

or a salt thereof, wherein: each of L^{G1} and L^{G2} s independently, a covalent bond, an amide bond, optionally substituted alkylene, optionally substituted alkenylene, optionally substituted heteroalkylene, optionally substituted heterocyclyldiyl, or optionally substituted (heterocyclyl)(alkyl)ene; and each of R^{G1} and R^{G2} is, independently, hydroxyl, optionally substituted hydroxyalkyl, optionally substituted hydroxyaryl, carboxyl, amino, optionally substituted aminoalkyl, optionally substituted aminoaryl, amido, optionally substituted cyclic anhydride, or optionally substituted cyclic imide.

Embodiment 6. The composition of embodiment 1, wherein the composition includes a structure having formula (Ib):

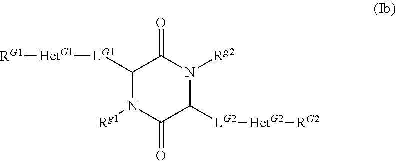

or a salt thereof, wherein: each of L^{G1} and L^{G2} is, independently, a covalent bond, an amide bond, optionally substituted alkylene, optionally substituted alkenylene, optionally substituted heteroalkylene, optionally substituted heterocyclyldiyl, or optionally substituted (heterocyclyl)(alkyl)ene; each of Het^{G1} and Het is, independently, optionally substituted heterocyclyldiyl or optionally substituted (heterocyclyl)(alkyl)ene; and each of R^{G1} and R^{G2} is, independently, hydroxyl, optionally substituted hydroxyalkyl, optionally substituted hydroxyaryl, carboxyl, amino, optionally substituted aminoalkyl, optionally substituted aminoaryl, amido, optionally substituted cyclic anhydride, or optionally substituted cyclic imide.

Embodiment 7. The composition of embodiment 1, wherein the composition includes a structure having formula (Ic):

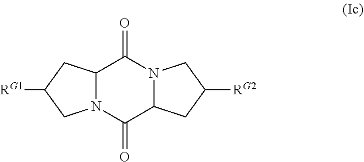

or a salt thereof, wherein: each of R^{G1} and R^{G2} is, independently, hydroxyl, optionally substituted hydroxyalkyl, carboxyl, amino, optionally substituted aminoalkyl, amido, optionally substituted cyclic anhydride, or optionally substituted cyclic imide.

Embodiment 8. A method of making a composition of embodiment 1, the method including: providing a first amino acid and a second amino acid, wherein the first and second amino acids are selected from the group consisting of glycine, vinylglycine, 2-allylglycine, alanine, valine, leucine, isoleucine, methionine, phenylalanine, 4-aminophenylalanine, tyrosine, tryptophan, serine, threonine, asparagine, glutamine, cystine, homocystine, cysteine, homocysteine, selenocysteine, proline, hydroxyproline, arginine, histidine, lysine, aspartic acid, glutaminic acid, 4-(2-amino-2-carboxyethyl)-1,2-benzenedicarboxylic acid, 1-aminopropane-1,2,3-tricarboxylic acid, and 4-amino-1,2,4-butanetricarboxylic acid; and forming a dimer between the first and second amino acids.

Embodiment 9. The method of embodiment 8, wherein the first and second amino acids are selected from the group consisting of tyrosine, serine, threonine, and hydroxyproline.

Embodiment 10. A method of making a composition of embodiment 1, the method including: providing a first amino acid and a second amino acid, wherein the first and second amino acids are selected from the group consisting of vinylglycine and 2-allylglycine; forming a dimer between the first and second amino acids; and hydroaminating a vinyl group present on the first and second amino acids.

Embodiment 11. A composition including a structure having formula (II):

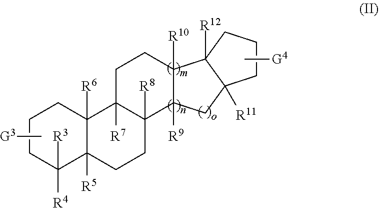

or a salt thereof, wherein: each of G³ and G⁴ includes, independently, hydroxyl, carboxyl, amino, amido, optionally substituted cyclic anhydride, or optionally substituted cyclic imide; each of R³, R⁴, R⁵, R⁶, R⁷, R⁸, R⁹, R¹⁰, R¹¹, and R¹² is, independently, H or optionally substituted alkyl; each of m and n is, independently, an integer of from 0 to 1; and o is an integer of from 0 to 2.

Embodiment 12. The composition of embodiment 11, wherein: G³ is -L^{G1}-R^{G1} and G⁴ is -L^{G2}-R^{G2}; each of L^{G1} and L^{G2} is, independently, a covalent bond, an amide bond, optionally substituted alkylene, optionally substituted alkenylene, optionally substituted heteroalkylene, optionally substituted arylene, optionally substituted (aryl)(alkyl)ene, optionally substituted heterocyclyldiyl, or optionally substituted (heterocyclyl)(alkyl)ene; and each of R^{G1} and R^{G2} is, independently, hydroxyl, optionally substituted hydroxyalkyl, optionally substituted hydroxyaryl, carboxyl, amino, optionally substituted aminoalkyl, optionally substituted aminoaryl, amido, optionally substituted cyclic anhydride, or optionally substituted cyclic imide.

Embodiment 13. A composition including a structure having formula (III):

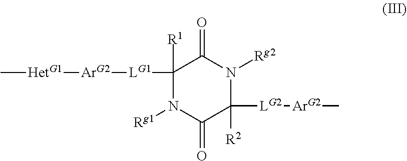

or a salt thereof, wherein: each of R¹ and R² is, independently, H or optionally substituted alkyl; each of R^{g1} and R^{g2} is, independently, optionally substituted alkyl, optionally substituted aryl, optionally substituted alkaryl, or optionally substituted aralkyl; each of L^{G1} and L^{G2} is, independently, a covalent bond, an amide bond, optionally substituted alkylene, optionally substituted alkenylene, optionally substituted heterocyclyldiyl, or optionally substituted (heterocyclyl)(alkyl)ene; each of Ar^{G1} and Ar^{G2} is, independently, optionally substituted arylene or optionally substituted (aryl)(alky)lene; Het^{G1} is optionally substituted heterocyclyldiyl or optionally substituted (heterocyclyl)(alkyl)ene; and at least one of Ar^{G1}, Ar^{G2}, or Het^{G1} includes, independently, hydroxyl, carboxyl, amino, amido, an amide bond, an ester bond, oxy, carbonyl, optionally substituted cyclic anhydride, or optionally substituted cyclic imide.

Embodiment 14. A composition including a structure having formula (IV):

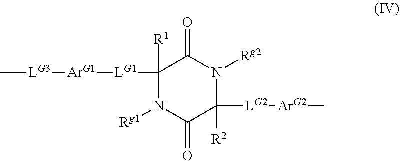

or a salt thereof, wherein: each of R¹ and R² is, independently, H or optionally substituted alkyl; each of R^{g1} and R^{g2} is, independently, optionally substituted alkyl, optionally substituted aryl, optionally substituted alkaryl, or optionally substituted aralkyl; each of L^{G1}, L^{G2}, and L^{G3} is, independently, a covalent bond, an amide bond, optionally substituted alkylene, optionally substituted alkenylene, optionally substituted heteroalkylene, optionally substituted heterocyclyldiyl, or (heterocyclyl)(alkyl)ene; each of Ar^{G1} and Ar^{G2} is, independently, optionally substituted arylene or optionally substituted (aryl)(alkyl)ene; and at least one of L^{G1} L^{G2} L^{G3}, Ar^{G1} or Ar^{G2} includes, independently, hydroxyl, carboxyl, amino, amido, an amide bond, an ester bond, oxy, carbonyl, optionally substituted cyclic anhydride, or optionally substituted cyclic imide.

Embodiment 15. A composition including a structure having formula (VIII):

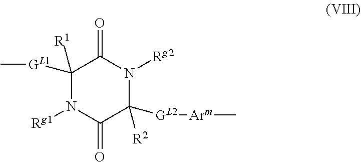

or a salt thereof, wherein: each of G^{L1} and G^{L2} includes, independently, hydroxyl, carboxyl, amino, amido, an amide bond, an ester bond, oxy, carbonyl, optionally substituted cyclic anhydride, or optionally substituted cyclic imide; each of R¹ and R² is, independently, H or optionally substituted alkyl; each of R^{g1} and R^{g2} is, independently, optionally substituted alkyl, optionally substituted aryl, optionally substituted alkaryl, or optionally substituted aralkyl; Ar^{m} is a polymer segment; R^{g1} and G¹, taken together with the nitrogen to which R^{g1} is bound, can optionally form an optionally substituted heterocyclyl; and R^{g2} and G², taken together with the nitrogen to which R^{g2} is bound, can optionally form an optionally substituted heterocyclyl.

Embodiment 16. The composition of embodiment 15, wherein Ar^{m} includes an imide subunit, an amic acid subunit, an amide subunit, an arylene subunit, an arylene ether subunit, an arylene ketone subunit, a urethane subunit, a phthalic anhydride subunit, an aliphatic subunit, a cycloalkyl subunit, an ether subunit, a thioether subunit, a perfluoroalkyl subunit, or a perfluoroalkoxy subunit.

Embodiment 17. The composition of embodiments 1-7 and 11-16, wherein the composition includes a UV cutoff less than about 450 nm, an optical transmission of at least about 95% at 450 nm, a yellowness index less than about 2, a modulus less than about 12 GPa, and/or an elongation less than about 350%.

Embodiment 18. The composition of embodiment 17, wherein the composition is optionally soluble in an organic solvent.

Embodiment 19. The composition of embodiments 1-7 and 11-18, wherein the composition is configured to be biodegradable by one or more microbes.

Embodiment 20. A genetically modified organism configured to produce a composition of embodiments 1-7 and 11-19.

Embodiment 21. A film including a composition of embodiments 1-7 and 11-19.

Embodiment 22. The film of embodiment 21, wherein the film is an adhesive or a coating.

Embodiment 23. A composite or bulk structure including a composition of embodiments 1-7 and 11-19.

Embodiment 24. A fiber or a particle including a composition of embodiments 1-7 and 11-19.

The following examples are provided to better disclose and teach processes and compositions of the present invention. They are for illustrative purposes only, and it must be acknowledged that minor variations and changes can be made without materially affecting the spirit and scope of the invention as recited in the claims that follow.

### EXAMPLES

### Example 1: Polymers and Copolymers from Amino Acid Dimers

The composition herein can be employed to provide a polymer or copolymer. In one instance, amino acids are employed to provide cyclic derivatives, which can be further functionalized with a reactive group containing hydroxy, amino, cyclic anhydride, or cyclic imide.

In one instance, the copolymer results from reaction between (i) a cyclic derivative having amino reactive groups and (ii) a dianhydride or a diacid. In another instance, the copolymer results from reaction between (i) a cyclic derivative having cyclic anhydride reactive groups and (ii) a diamine. In yet another instance, the copolymer results from reaction between (i) a cyclic derivative having cyclic anhydride reactive groups and (ii) a cyclic derivative having amino reactive groups. Resulting copolymers can have or include structures of compound nos. VIII-1 to VIII-9 in Table 2, as well as structures of formulas (III), (IIIa), (IV), (IVa), (IVb), (IVc), (V), (Va), (VI), (VIa), (VIb), (VIc), (VII), (VIIa), and (VIII).

### Example 2: Non-Limiting Synthesis of Tyrosine Dimer

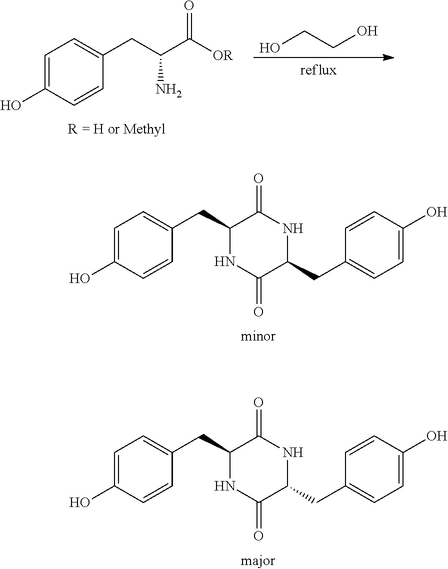

In a 3 L two-neck round bottom flask equipped with magnetic stirrer and overhead condenser, 200 g of Tyr-OH and 800 ml of ethylene glycol were mixed, and the flask was placed in silicon oil bath. The oil bath was heated to 190° C., and the reaction mixture was stirred for 7 hours (h). The conversion of starting material was followed up by HPLC. After 7 h, the reaction mixture was cooled down to room temperature, and the precipitated solid was filtered and washed with ethanol (2×200 ml). The solid was then dried in vacuum oven and used as is for the next step. (Yield: 64%)

### Example 3: Non-Limiting Synthesis of 4-Hydroxy-Proline Dimer

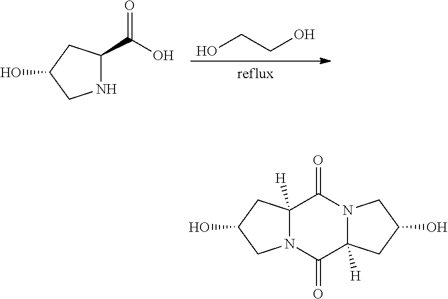

In a two-neck 1 L round bottom flask equipped with magnetic stirrer and overhead condenser, 100 g of trans-4-hydroxy-L-proline and 200 ml of ethylene glycol were mixed, and the flask was placed in silicon oil bath. The oil bath was heated to 190° C., and the reaction mixture was stirred for 7 h. After 7 h, the reaction mixture was cooled down to room temperature, and the precipitated solid was filtered and washed with acetone (2×100 ml). The solid was then dried in vacuum oven. (Yield: 44%, isolated 37.95 grams of product) NMR ¹H NMR (D₂O): 4.75 (d, 1H), 4.63 (d, 1H), 3.69 (d, 1H), 3.537 (d, 1H), 2.33 (d, 1H), 2.20 (d, 1H).

### Example 4: Non-Limiting Stepwise Synthesis of Tyrosine Dimer

The following route could be applicable for dimers from different amino acids.

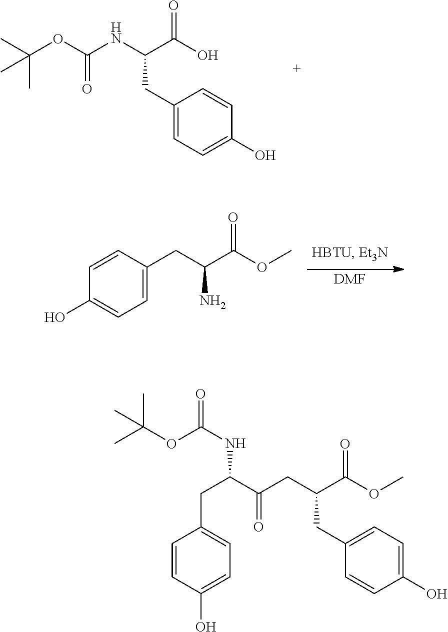

### Step 1: Preparation of (S)-methyl 2-((R)-2-((tert-butoxycarbonyl)amino)-3-(4-hydroxyphenyl) propanamido)-3-(4-hydroxyphenyl)propanoate

A 1 L reactor equipped with a magnetic stirrer, temperature probe, and nitrogen inlet was charged with ((S)-2-((tert-butoxycarbonyl)amino)-3-(4-hydroxyphenyl)propanoic acid (33.2 g, 118 mmol), (S)-methyl 2-amino-3-(4-hydroxyphenyl)propanoate (20 g, 102 mmol), hexafluorophosphate benzotriazole tetramethyl uronium (“HBTU,” 48.3 g, 127 mmol) and DMF (120 mL). The solution was stirred for 15 minutes and then cooled to 0° C. Triethylamine (42.6 mL, 306 mmol) was added to the mixture over 15 minutes. After the addition was completed, the cooling bath was removed, and the reaction was stirred overnight. After 18 h, the HPLC of the aliquot showed complete conversion of the starting materials. One hundred mL of water was slowly added to the reaction at 0° C. After stirring for 30 minutes (min), the mixture was diluted with EtOAc (150 mL), and the layers were separated. The organic layer was washed with aqueous sodium carbonate (10%, 3×50 mL) and finally with brine (50 mL). The organic layer was then dried over anhydrous sodium sulfate, filtered, and concentrated to dryness to afford the desired product as a thick oil. The product was used in the next step without further purification.

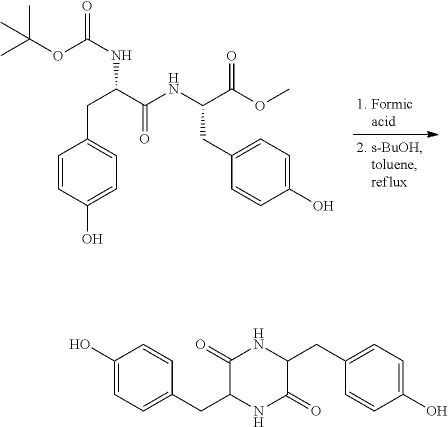 [SMILES: COC(=O)[C@H](Cc1ccc(O)cc1)NC(=O)[C@H](Cc1ccc(O)cc1)NC(=O)OC(C)(C)C ; O=C1NC(Cc2ccc(O)cc2)C(=O)NC1Cc1ccc(O)cc1]

### Step 2: Preparation of 3,6-bis(4-hydroxybenzyl)piperazine-2,5-dione

A 3 L single-neck reactor was charged with (S)-methyl 2-((R)-2-((tertbutoxycarbonyl)amino)-3-(4-hydroxyphenyl)propanamido)-3-(4-hydroxyphenyl)propanoate (42 g, 91.6 mmol) and formic acid (420 mL), the mixture was stirred at ambient temperature for 5 h, and the formic acid and s-butanol were removed under reduced pressure. The residue was dissolved in sec-butanol (1600 mL) and toluene (400 mL), and the solution was refluxed for 3 h. The reaction was monitored by HPLC and, after the reaction was completed, the reaction mixture was concentrated to yield the crude material as an off-white solid. The crude material was dissolved in 5% NaOH in water at 5° C. and extracted with 250 ml of ethyl acetate. The aqueous layer was acidified to pH 3 by the slow addition of 10% HCl (aq). The solid material was separated by filtration, washed with water, and dried under vacuum. The solid was suspended in 200 ml of acetonitrile and filtered again and dried to get a white solid as a pure product. (Yield: 22 g, 73%). NMR H NMR (DMSO): 9.20 (s, 1H), 7.76 (s, 1H), 6.84 (d, J=8.4 Hz, 2H), 6.67 (d, J=8.5 Hz, 2H), 3.85 (s, 1H), 2.55-2.51 (m, 1H), 2.12 (d, J=6.6 Hz, 1H).

### Example 5: Non-Limiting Stepwise Synthesis of p-Hydroxyphenyl-Glycine Dimer

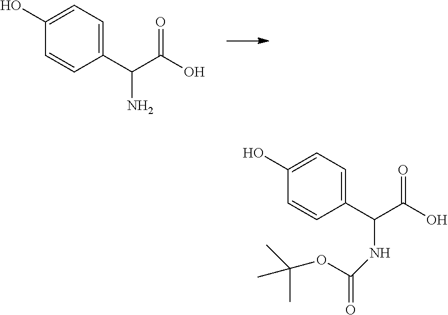 [SMILES: NC(C(=O)O)c1ccc(O)cc1 ; CC(C)(C)OC(=O)NC(C(=O)O)c1ccc(O)cc1]

(2R)-2-Amino-2-(4-hydroxyphenyl)acetic acid (1.00 eq, 1.00 g, 5.98 mmol) was dissolved in 1,4-dioxane (24 mL), water (24 ml), and 12.5 ml of an aqueous 2M NaOH solution in a 100 ml 2 neck flask under nitrogen. Di-tert-butyl dicarbonate (1.00 eq, 1.31 g, 5.98 mmol) was added to the solution dropwise, and the reaction was allowed to stir for 16 hours at room temperature. The reaction mixture was concentrated then acidified to pH 2 with 5M HCl, then extracted with ethyl acetate, and washed with a 5% sodium carbonate solution and brine. The organic layers were dried over magnesium sulfate, filtered, then concentrated in vacuo. Finally, 2-(tert-butoxycarbonylamino)-2-(4-hydroxyphenyl)acetic acid [4-HPG N-Boc](1.08 g, 4.03 mmol, 67.36% yield) was isolated as a pink tacky solid and used in the next step without further purification.

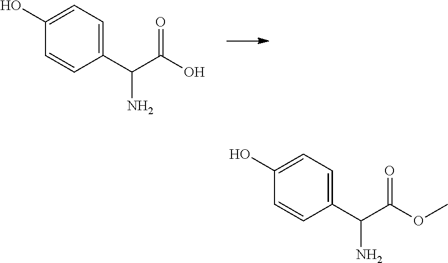

rac-(2R)-2-amino-2-(4-hydroxyphenyl)acetic acid (1.00 eq, 1.00 g, 5.98 mmol) was dissolved in 20 ml of 1.25M HCl in methanol and stirred at 70° C. for 3 hours. Then, the solvent was evaporated on a rotovap to yield 1.064 g of crude pink-white solid. This solid was washed with 250 ml of saturated sodium carbonate and extracted with ethyl acetate (4×100 ml) to provide 4-hydroxyphenyl-glycine methyl ester [4-HPG OMe]. (Yield: 33.766%, isolated 0.366 g of product).

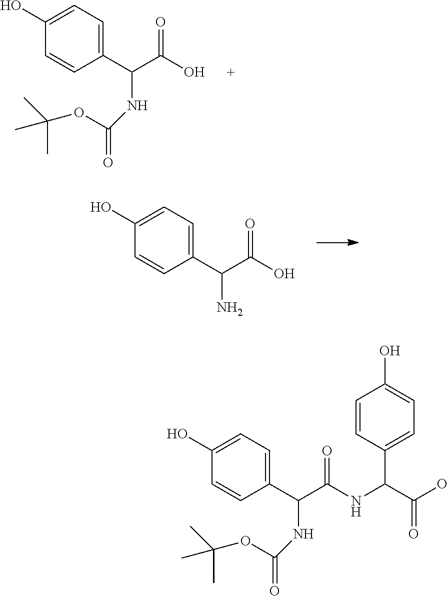

4-hydroxyphenyl-glycine methyl ester (4-HPG OMe), 4-HPG N-Boc, HBTU, and DMAc were added to a 25 ml 2 neck round bottom flask and stirred for 15 min at room temperature under nitrogen. The reaction was cooled to 0° C., and trimethylamine (0.70 ml) was added dropwise over 15 min and then allowed to stir overnight. The reaction was then quenched with 2 ml of ice cold water, stirred for 10 mins and extracted 3× with EtoAc (2 ml). The organic layers were washed with 5% sodium carbonate and then brine, dried, and concentrated.

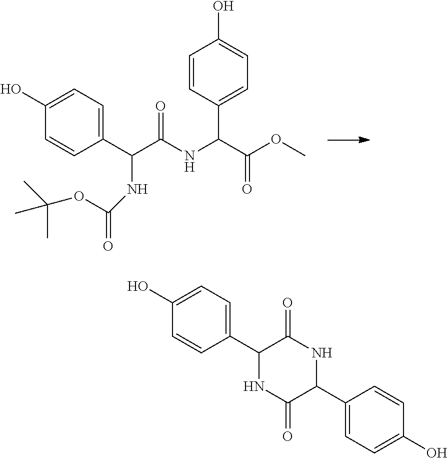 [SMILES: COC(=O)C(NC(=O)C(NC(=O)OC(C)(C)C)c1ccc(O)cc1)c1ccc(O)cc1 ; O=C1NC(c2ccc(O)cc2)C(=O)NC1c1ccc(O)cc1]

A 3 L single-neck reactor was charged with the foregoing dipeptide peptide (0.31 g) and formic acid (2.1 mL), and the mixture was stirred at ambient temperature for 5 h. Formic acid was removed under reduced pressure by azeotropic distillation with toluene. The residue was dissolved in sec-butanol (7.5 mL) and toluene (2.5 mL), and the solution was refluxed for 3 hours. The reaction mixture was concentrated to yield the crude material as a yellow-white solid.

### Example 6: Non-Limiting Stepwise Synthesis of 3,20-Diamino-Pregnane

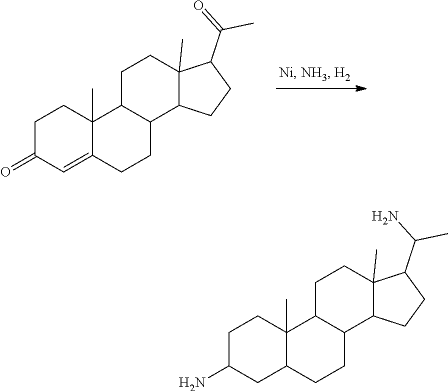 [SMILES: CC(=O)C1CCC2C3CCC4=CC(=O)CCC4(C)C3CCC12C ; CC(N)C1CCC2C3CCC4CC(N)CCC4(C)C3CCC12C]

Progesterone (1 g, 3.1 mmol) and Raney nickel (1 g) were added to a 100 ml high pressure hydrogenation apparatus and the vessel was charged with liquid ammonia (25 ml) and the vessel was sealed. The total vessel pressure was then increased to 25 atmospheres and the reaction vessel was heated at 200° C. for 20 h. The vessel was subsequently cooled and the pressure released. Upon evaporation of the ammonia, the mixture was dissolved in methanol and the catalyst removed. The reside was purified by column chromatography to afford 70% yield of the target 3,20-diamino pregnane (700 mg).

### Example 7: Non-Limiting Stepwise Synthesis of 4-Aminoproline Dimer

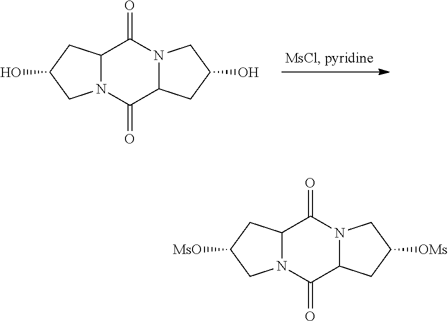 [SMILES: O=C1C2C[C@@H](O)CN2C(=O)C2C[C@@H](O)CN12 ; CS(=O)(=O)O[C@@H]1CC2C(=O)N3C[C@H](OS(C)(=O)=O)CC3C(=O)N2C1]

The 4-hydroxy-proline dimer (20 g, 88 mmol) was suspended in pyridine (60 ml) and a catalytic amount of dimethyl aminopyridine (600 mg) was added. The mixture was cooled to −10° C. and Methanesulfonyl chloride (16 ml, 200 mmol) was added dropwise. The mixture was allowed to warm to room temperature and was stirred for 20 h at 20° C. before being poured over dilute HCl in ice water. The resulting solid was collected and dried under vacuum to give 81% yield (27 g) of the target dimesyl-4-hydroxy proline dimer.

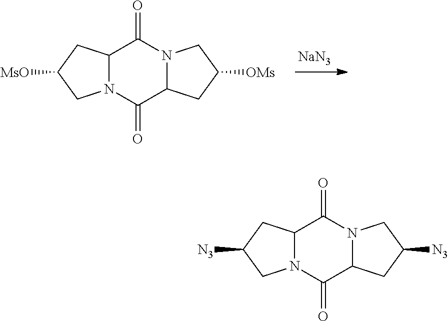 [SMILES: CS(=O)(=O)O[C@@H]1CC2C(=O)N3C[C@H](OS(C)(=O)=O)CC3C(=O)N2C1 ; [N-]=[N+]=N[C@H]1CC2C(=O)N3C[C@@H](N=[N+]=[N-])CC3C(=O)N2C1 ; "NaN3"]

The dimesyl-4-hydroxy proline dimer (25 g, 65 mmol) was dissolved in DMF (250 ml) and cooled to 0° C. Sodium azide (21.5 g, 327 mmol) was added slowly and the reaction mixture was heated to 75° C. and stirred under a nitrogen atmosphere for 72 h. The mixture was cooled, diluted with ethyl acetate (500 ml), filtered, and concentrated to give 17.5 g of crude diazide for the next step.

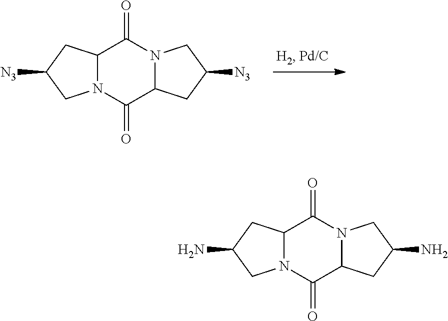

The hydroxy-proline dimer based diazide (10 g, 36 mmol) was dissolved in methanol (100 ml) and added to palladium on carbon (1 g) in a hydrogenation reactor. The atmosphere was replaced with hydrogen and the reactor was charged to 5 atmospheres of pressure. The mixture was allowed to stir under hydrogen atmosphere for 4 hours. The pressure was released, the catalysts filtered through celite and the solvent was removed in vacuo to afford the target 4-amino-proline dimer in 96% yield (7.7 g).

### Example 8: Non-Limiting Stepwise Synthesis of 4-Amino-Phenylalanine Dimer

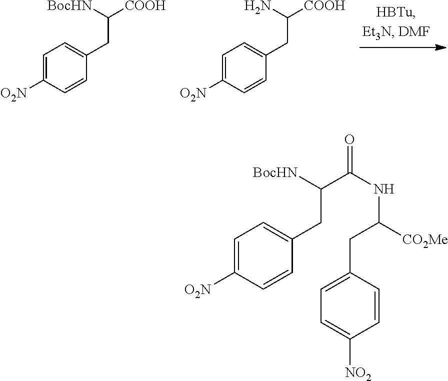 [SMILES: CC(C)(C)OC(=O)NC(Cc1ccc([N+](=O)[O-])cc1)C(=O)O ; NC(Cc1ccc([N+](=O)[O-])cc1)C(=O)O ; COC(=O)C(Cc1ccc([N+](=O)[O-])cc1)NC(=O)C(Cc1ccc([N+](=O)[O-])cc1)NC(=O)OC(C)(C)C]

4-Nitrophenylalanine methyl ester (14.3 g, 64 mmol) and 4-nitro-N-(tert-Butoxycarbonyl)-phenylalanine (20 g, 64 mmol) were combined in DMF (500 ml). HBTU (29 g, 77 mmol) was added and the mixture was stirred for 15 min and cooled to 0° C. Triethylamine (50 ml) was added slowly at 0° C. and the mixture was allowed to warm to room temperature and stirred for 20 h. The reaction mixture was quenched with cold water and the target dipeptide extracted using ethyl acetate and used for the subsequent step without purification.

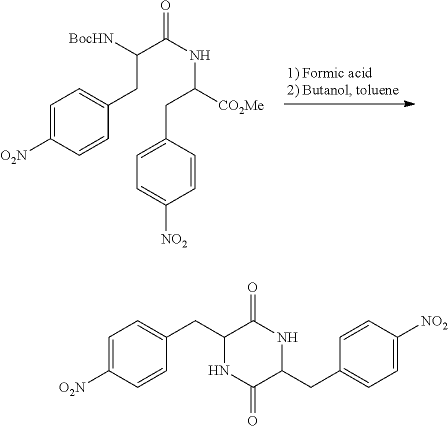 [SMILES: COC(=O)C(Cc1ccc([N+](=O)[O-])cc1)NC(=O)C(Cc1ccc([N+](=O)[O-])cc1)NC(=O)OC(C)(C)C ; O=C1NC(Cc2ccc([N+](=O)[O-])cc2)C(=O)NC1Cc1ccc([N+](=O)[O-])cc1]

The dinitro-dipeptide (34 g, 66 mmol) was suspended in formic acid (300 ml) and stirred for 18 h at room temperature. Then 2-butanol (3.5 L) and toluene (850 ml) were added and the temperature was raised to 100° C. and the mixture was stirred for 4 h. The mixture was then cooled and the resulting nitro-phenylalanine dimer was filtered off and washed with methanol and dried to give the target compound in 72% yield (18 g).

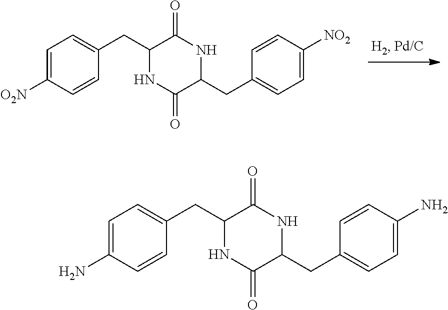 [SMILES: O=C1NC(Cc2ccc([N+](=O)[O-])cc2)C(=O)NC1Cc1ccc([N+](=O)[O-])cc1 ; Nc1ccc(CC2NC(=O)C(Cc3ccc(N)cc3)NC2=O)cc1]

The nitro-phenylalanine dimer (12 g, 31 mmol) and palladium on carbon (1.2 g) was suspended in 10% HCl in methanol (1 L) in a hydrogenation vessel. The vessel was charged to 50 psi and the mixture was stirred for 8 hours. The pressure was then released and the mixture filtered to remove the catalyst. The solvent volume was reduced and the solid isolated and treated with base to generate the target molecule.

### Example 9: Non-Limiting Stepwise Synthesis of 3-Aminophenyl-Glycine Dimer

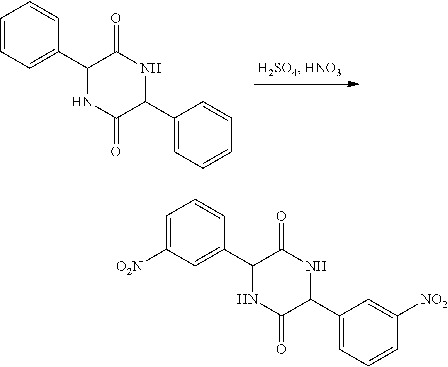

The phenyl-glycine dimer (39 g, 25 mmol) was suspended in Sulfuric acid (50 ml) and cooled to 10° C. To this was added a 1:1 mixture of fuming nitric acid and sulfuric acid (50 ml) dropwise. The mixture was allowed to stir for 4 hours and then was poured over ice. The resulting solid was filtered to give 50 g of solid that was washed copiously with methanol.

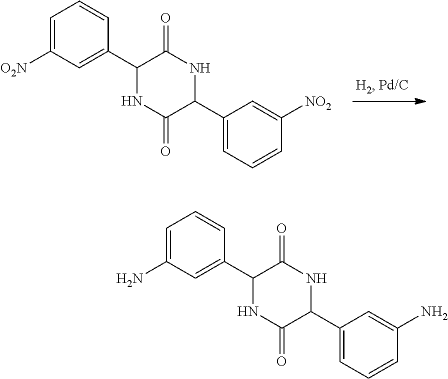 [SMILES: O=C1NC(c2cccc([N+](=O)[O-])c2)C(=O)NC1c1cccc([N+](=O)[O-])c1 ; Nc1cccc(C2NC(=O)C(c3cccc(N)c3)NC2=O)c1]

The nitro-phenyl-glycine dimer (12 g, 34 mmol) and palladium on carbon (1 g) was suspended in ethanol (50 ml) containing 10% HCl. This mixture was added to a hydrogenation vessel and the system was charged to 50 psi and allowed to stir for 20 h. After the pressure was released, the catalyst was filtered off and the neutralization of the filtrate gave 7.9 g of the meta-amino-phenylglycine as a solid. The resulting compound was recrystallized from DMF.

### Example 10: Non-Limiting Stepwise Synthesis Diamino-Estrane

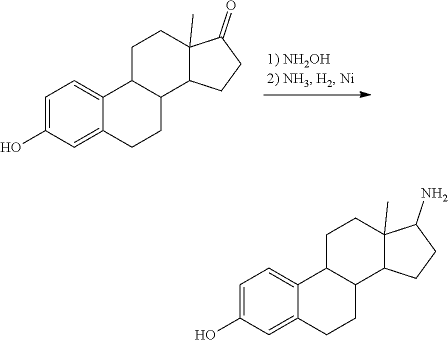 [SMILES: CC12CCC3c4ccc(O)cc4CCC3C1CCC2=O ; CC12CCC3c4ccc(O)cc4CCC3C1CCC2N]

Estrone (15 g, 56 mmol) and potassium carbonate (15 g, 110 mmol) were suspended in ethanol (50 ml), hydroxylamine hydrochloride (5.5 g, 80 mmol) was added in small portions and the mixture was allowed to stir for 2 hours before being dumped over ice water. The resulting solid was filtered to 11 g of intermediate for the next step. The oxamine intermediate (9 g, 32 mmol) was dissolved in 25% ammonia in methanol (300 ml) and Raney Nickel (5 g) was added and the mixture was transferred to a hydrogenation vessel. The system was then charged to 50 atmospheres and stirred at 40° C. for 20 hours. The mixture was then cooled, the pressure released, and the catalyst filtered away to give the hydroxy-amino-estrone intermediate in 93% yield (8 g).

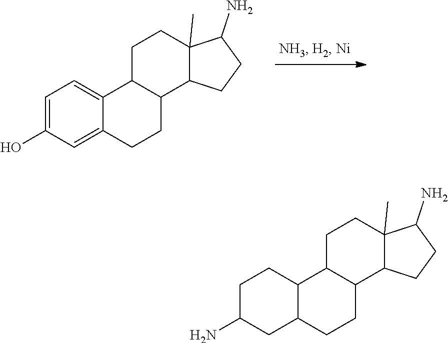 [SMILES: CC12CCC3c4ccc(O)cc4CCC3C1CCC2N ; CC12CCC3C4CCC(N)CC4CCC3C1CCC2N]

The hydroxy-amino-estrone (2.2 g, 8.2 mmol) produced in the previous step was charged into a hydrogenation vessel with Raney Nickel (2 g) and liquid ammonia (20 ml) was added. The vessel pressure was increased to 50 atmospheres with hydrogen and the system was heated to 200° C. for 20 h. Upon cooling, the pressure was released and the mixture was dissolved in methanol and the catalyst was removed using filtration and evaporation gave the target compound in 84% yield as a mixture of isomers (1.7 g).

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

1. A composition comprising a structure having formula (I): 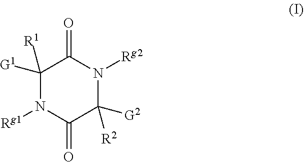
or a salt thereof, wherein:
each of G¹ and G² comprises, independently, hydroxyl, carboxyl, amino, amido, optionally substituted cyclic anhydride, or optionally substituted cyclic imide;
each of R¹ and R² is, independently, H or optionally substituted alkyl;
each of R^{g1} and R^{g2} is, independently, optionally substituted alkyl, optionally substituted aryl, optionally substituted alkaryl, or optionally substituted aralkyl;
R^{g1} and G¹, taken together with the nitrogen to which R^{g1} is bound, can optionally form an optionally substituted heterocyclyl; and
R^{g2} and G², taken together with the nitrogen to which R^{g2} is bound, can optionally form an optionally substituted heterocyclyl.

2. The composition of claim 1, wherein:
G¹ is -L^{G1}-R^{G1} and G² is -L^{G2}-R^{G2};
each of L^{G1} and L^{G2} is, independently, a covalent bond, an amide bond, optionally substituted alkylene, optionally substituted alkenylene, optionally substituted heteroalkylene, optionally substituted heterocyclyldiyl, or optionally substituted (heterocyclyl)(alkyl)ene; and
each of R^{G1} and R^{G2} is, independently, hydroxyl, optionally substituted hydroxyalkyl, optionally substituted hydroxyaryl, carboxyl, amino, optionally substituted aminoalkyl, optionally substituted aminoaryl, amido, optionally substituted amidoalkyl, optionally substituted cyclic anhydride, or optionally substituted cyclic imide.

3. The composition of claim 2, wherein the optionally substituted cyclic anhydride has a structure of: 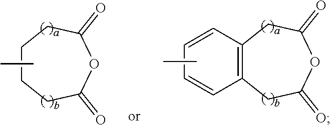
and wherein each of a and b, independently, is an integer of from 0 to 3.

4. The composition of claim 2, wherein the optionally substituted cyclic imide has a structure of: 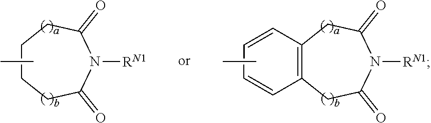
wherein each of a and b, independently, is an integer of from 0 to 3; and wherein R^{N1} is H, optionally substituted alkyl, optionally substituted aryl, or optionally substituted aralkyl.

5. The composition of claim 1, wherein the composition comprises a structure having formula (Ia): 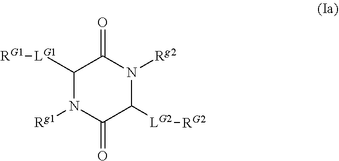
or a salt thereof, wherein:
each of L^{G1} and L^{G2} is, independently, a covalent bond, an amide bond, optionally substituted alkylene, optionally substituted alkenylene, optionally substituted heteroalkylene, optionally substituted heterocyclyldiyl, or optionally substituted (heterocyclyl)(alkyl)ene; and
each of R^{G1} and R^{G2} is, independently, hydroxyl, optionally substituted hydroxyalkyl, optionally substituted hydroxyaryl, carboxyl, amino, optionally substituted aminoalkyl, optionally substituted aminoaryl, amido, optionally substituted cyclic anhydride, or optionally substituted cyclic imide.

6. The composition of claim 1, wherein the composition comprises a structure having formula (Ib): 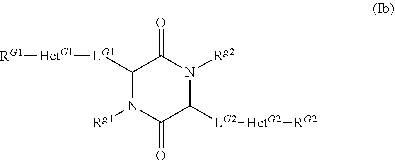
or a salt thereof, wherein:
each of L^{G1} and L^{G2} is, independently, a covalent bond, an amide bond, optionally substituted alkylene, optionally substituted alkenylene, optionally substituted heteroalkylene, optionally substituted heterocyclyldiyl, or optionally substituted (heterocyclyl)(alkyl)ene;
each of Het^{G1} and Het^{G2} is, independently, optionally substituted heterocyclyldiyl or optionally substituted (heterocyclyl)(alkyl)ene; and
each of R^{G1} and R^{G2} is, independently, hydroxyl, optionally substituted hydroxyalkyl, optionally substituted hydroxyaryl, carboxyl, amino, optionally substituted aminoalkyl, optionally substituted aminoaryl, amido, optionally substituted cyclic anhydride, or optionally substituted cyclic imide.

7. The composition of claim 1, wherein the composition comprises a structure having formula (Ic) 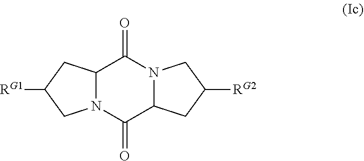
or a salt thereof, wherein:
each of R^{G1} and R^{G2} is, independently, hydroxyl, optionally substituted hydroxyalkyl, carboxyl, amino, optionally substituted aminoalkyl, amido, optionally substituted cyclic anhydride, or optionally substituted cyclic imide.

8. (canceled)

9. (canceled)

10. (canceled)

11. A composition comprising a structure having formula (II): 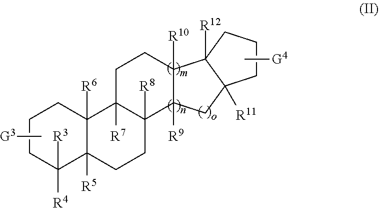
or a salt thereof, wherein:
each of G³ and G⁴ comprises, independently, hydroxyl, carboxyl, amino, amido, optionally substituted cyclic anhydride, or optionally substituted cyclic imide;
each of R³, R⁴, R⁵, R⁶, R⁷, R⁸, R⁹, R¹⁰, R¹¹, and R¹² is, independently, H or optionally substituted alkyl;
each of m and n is, independently, an integer of from 0 to 1; and
o is an integer of from 0 to 2.

12. The composition of claim 11, wherein:
G³ is -L^{G1}-R^{G1} and G⁴ is -L^{G2}-R^{G2};
each of L^{G1} and L^{G2} is, independently, a covalent bond, an amide bond, optionally substituted alkylene, optionally substituted alkenylene, optionally substituted heteroalkylene, optionally substituted arylene, optionally substituted (aryl)(alkyl)ene, optionally substituted heterocyclyldiyl, or optionally substituted (heterocyclyl)(alkyl)ene; and
each of R^{G1} and R^{G2} is, independently, hydroxyl, optionally substituted hydroxyalkyl, optionally substituted hydroxyaryl, carboxyl, amino, optionally substituted aminoalkyl, optionally substituted aminoaryl, amido, optionally substituted cyclic anhydride, or optionally substituted cyclic imide.

13. A composition comprising a structure having formula (III): 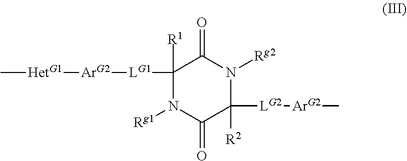
or a salt thereof, wherein:
each of R¹ and R² is, independently, H or optionally substituted alkyl;
each of R^{g1} and R^{g2} is, independently, optionally substituted alkyl, optionally substituted aryl, optionally substituted alkaryl, or optionally substituted aralkyl;
each of L^{G1} and L^{G2} is, independently, a covalent bond, an amide bond, optionally substituted alkylene, optionally substituted alkenylene, optionally substituted heterocyclyldiyl, or optionally substituted (heterocyclyl)(alkyl)ene;
each of Ar^{G1} and Ar^{G2} is, independently, optionally substituted arylene or optionally substituted (aryl)(alky)lene;
Het^{G1} is optionally substituted heterocyclyldiyl or optionally substituted (heterocyclyl)(alkyl)ene; and
at least one of Ar^{G1}, Ar^{G2}, or Het^{G1} comprises, independently, hydroxyl, carboxyl, amino, amido, an amide bond, an ester bond, oxy, carbonyl, optionally substituted cyclic anhydride, or optionally substituted cyclic imide.

14. A composition comprising a structure having formula (IV): 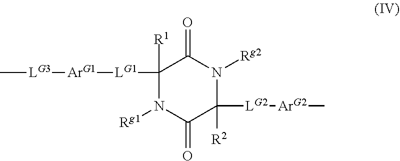
or a salt thereof, wherein:
each of R¹ and R² is, independently, H or optionally substituted alkyl;
each of R^{g1} and R^{g2} is, independently, optionally substituted alkyl, optionally substituted aryl, optionally substituted alkaryl, or optionally substituted aralkyl;
each of L^{G1}, L^{G2}, and L^{G3} is, independently, a covalent bond, an amide bond, optionally substituted alkylene, optionally substituted alkenylene, optionally substituted heteroalkylene, optionally substituted heterocyclyldiyl, or (heterocyclyl)(alkyl)ene;
each of Ar^{G1} and Ar^{G2} is, independently, optionally substituted arylene or optionally substituted (aryl)(alkyl)ene; and
at least one of L^{G1}, L^{G2}, L^{G3}, Ar^{G1} or Ar^{G2} comprises, independently, hydroxyl, carboxyl, amino, amido, an amide bond, an ester bond, oxy, carbonyl, optionally substituted cyclic anhydride, or optionally substituted cyclic imide.

15. A composition comprising a structure having formula (VIII): 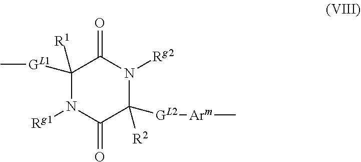
or a salt thereof, wherein:
each of G^{L1} and G^{L2} comprises, independently, hydroxyl, carboxyl, amino, amido, an amide bond, an ester bond, oxy, carbonyl, optionally substituted cyclic anhydride, or optionally substituted cyclic imide;
each of R¹ and R² is, independently, H or optionally substituted alkyl;
each of R^{g1} and R^{g2} is, independently, optionally substituted alkyl, optionally substituted aryl, optionally substituted alkaryl, or optionally substituted aralkyl;
Ar^{m} is a polymer segment;
R^{g1} and G¹, taken together with the nitrogen to which R^{g1} is bound, can optionally form an optionally substituted heterocyclyl; and
R^{g2} and G², taken together with the nitrogen to which R^{g2} is bound, can optionally form an optionally substituted heterocyclyl.

16. The composition of claim 15, wherein Ar^{m} comprises an imide subunit, an amic acid subunit, an amide subunit, an arylene subunit, an arylene ether subunit, an arylene ketone subunit, a urethane subunit, a phthalic anhydride subunit, an aliphatic subunit, a cycloalkyl subunit, an ether subunit, a thioether subunit, a perfluoroalkyl subunit, or a perfluoroalkoxy subunit.

17. The composition of claim 1, wherein the composition comprises a UV cutoff less than about 450 nm, an optical transmission of at least about 95% at 450 nm, a yellowness index less than about 2, a modulus less than about 12 GPa, and/or an elongation less than about 350%.

18. The composition of claim 17, wherein the composition is optionally soluble in an organic solvent.

19. The composition of claim 1, wherein the composition is configured to be biodegradable by one or more microbes.

20. A genetically modified organism configured to produce a composition of claim 1.

21. A film comprising a composition of claim 1.

22. (canceled)

23. A composite or bulk structure comprising a composition of claim 1.

24. A fiber or a particle comprising a composition of claim 1.
